(19) United States
(12) Patent Application Publication
(10) Pub. No.: US 2026/0348975 A1
(43) Pub. Date: Nov. 5, 2026

(54) ASSAY FOR DETECTION OF IL-33

(71) Applicant: MEDIMMUNE LIMITED, Cambridge (GB)

(72) Inventors: Emma Suzanne COHEN, Cambridge (GB); Darren Jonathan SCHOFIELD, Cambridge (GB); Ian Christopher SCOTT, Cambridge (GB); Elizabeth Helen ENGLAND, Cambridge (GB); Jennifer Louise PERCIVAL-ALWYN, Cambridge (GB)

(21) Appl. No.: 19/104,830

(22) PCT Filed: Aug. 18, 2023
(86) PCT No.: PCT/EP2023/072812

§ 371 Date: Feb. 19, 2025

Related U.S. Application Data
(60) Provisional application No. 63/371,932, filed on Aug. 19, 2022.
(60) Provisional application No. 63/385,817, filed on Dec. 2, 2022.

(51) Int. Cl. G01N 33/68; C07K 16/24

(52) U.S. Cl. CPC G01N 33/6869; C07K 16/244; C07K 2317/92; G01N 2333/54

(57) ABSTRACT

The present disclosure relates to a method for detecting antigenically distinct forms of IL-33 in a biological sample, kits for performing such methods, binding molecules and reporter molecules for use in such methods.

### CROSS-REFERENCE TO RELATED APPLICATIONS

This application claims benefit under 35 U.S.C. § 119(e) of U.S. Provisional Patent Application No. 63/371,932, filed Aug. 19, 2022, and U.S. Provisional Patent Application No. 63/385,817, filed Dec. 2, 2022, and which are incorporated by reference herein in their entirety for all purposes.

### REFERENCE TO SEQUENCE LISTING SUBMITTED ELECTRONICALLY

This application incorporates by reference a Sequence Listing submitted with this application in computer readable form (CRF) as a text file entitled “IL33-208-WO-PCT Sequence Listing” created on Aug. 17, 2023 and having a size of 46,433 bytes.

### FIELD

The present disclosure relates to a method for detecting antigenically distinct forms of IL-33 in a biological sample, kits for performing such methods, binding molecules and reporter molecules for use in such methods.

### BACKGROUND

Interleukin-33 (IL-33), also known as IL-1F11, is a member of the IL-1 family of cytokines. IL-33 is a 270 amino acid protein consisting of two domains: a homeodomain and a cytokine (IL-1 like) domain. The homeodomain contains a nuclear localisation signal (NLS). IL-33 is known to exist in different forms; a reduced form (redIL-33) and an oxidised form (oxIL-33). Previous studies have shown that the reduced form is rapidly oxidised under physiological conditions to form at least one disulphide bond in the oxidised form, and that the two forms likely have different binding patterns and effects.

It was previously discovered that the reduced form of IL-33 binds to ST2, and is in fact the only known ligand of the ST2 receptor expressed by Th2 cells and mast cells. Reduced IL-33 stimulates target cells by binding ST2 and subsequently activating NFkB and MAP kinase pathways leading to production of cytokines and chemokines such as IL-4, IL-5 and IL-13 for promoting inflammation. Soluble ST2 (sST2) is thought to be a decoy receptor that prevents reduced-IL-33 signalling.

More recently, it was found that the oxidised form of IL-33 also has physiological effects. It was discovered that oxidised IL-33 does not bind ST2, but instead binds to the receptor for advanced glycation end products (RAGE) and signals through this alternative pathway.

Accordingly, there has been significant interest in IL-33 as a therapeutic target, predominantly due to the ability of what is now known as the reduced form to stimulate ST2 and result in potent inflammatory effects, but also now due to the ability of the oxidised form to stimulate RAGE which is involved in many wide ranging diseases.

Given the different forms of IL-33 that are now known to exist and the different physiological effects associated with each form, it would be beneficial to be able to detect such distinct forms in a biological sample.

However, until now, no means of reliably discriminating between the different forms of IL-33 have been available. Some assays have been developed which detect IL-33. However, read out from these assays typically does not distinguish between the different forms, and therefore cannot inform users about the biological pathways that may be inhibited or activated in a given biological sample. A user has no way of knowing if the results indicate activation of the ST2 pathway or the RAGE pathway, for example. Furthermore, because these assays do not distinguish between the different forms, it is not clear which form is being measured in any given assay, and the results often lack consistency and reliability.

The present inventors have developed a highly sensitive assay which can detect not only one, but each of the different forms of IL-33; reduced IL33, oxidised IL33, and also bound IL33/sST2 in a biological sample. The assay is based on the development of novel binding molecules that can bind to each of the different forms of IL-33. The inventors believe that such an assay will be useful to analyse the antigenically distinct forms of IL-33 present in a sample from a patient in order to inform medical treatments.

Accordingly, one or more aspects of the present disclosure and further features thereof are defined hereinbelow.

### Aspects of the Disclosure

According to a first aspect of the present disclosure there is provided a method for detecting one or more antigenically distinct forms of IL-33 in a biological sample, comprising:
(a) contacting the sample with one or more binding molecules each capable of binding to an antigenically distinct form of IL-33 selected from: reduced IL-33, oxidised IL-33, and/or IL-33/sST2, under conditions sufficient to form complexes; and
(b) detecting the levels of the one or more reduced IL-33, oxidised IL-33, and/or IL-33/sST2 complexes in said sample, wherein:
   (i) the binding molecule which binds to reduced IL-33 comprises:
      a. a variable heavy domain having VHCDRs 1-3 of SEQ ID NOs: 1, 2 and 3, respectively, and a variable light domain having VLCDRs 1-3 of SEQ ID NOs: 5, 6 and 7, respectively, wherein one or more CDRs have 3 or fewer single amino acid substitutions, deletions or insertions;
      b. a variable heavy domain having VHCDRs 1-3 of SEQ ID NOs: 25, 26 and 27, respectively, and a variable light domain having VLCDRs 1-3 of SEQ ID NOs: 29, 30 and 31, respectively, wherein one or more CDRs have 3 or fewer single amino acid substitutions, deletions or insertions; or
      c. a variable heavy domain having VHCDRs 1-3 of SEQ ID NOs: 35, 36 and 37, respectively, and a variable light domain having VLCDRs 1-3 of SEQ ID NOs: 39, 40 and 41, respectively, wherein one or more CDRs have 3 or fewer single amino acid substitutions, deletions or insertions;
   (ii) the binding molecule which binds to oxidised IL-33 comprises a variable heavy domain having VHCDRs 1-3 of SEQ ID NOs: 9, 10 and 11, respectively, and a variable light domain having VLCDRs 1-3 of SEQ ID NOs: 13, 14 and 15, respectively wherein one or more CDRs have 3 or fewer single amino acid substitutions, deletions or insertions; and
   (iii) the binding molecule which binds to IL-33/ST2 comprises a variable heavy domain having VHCDRs 1-3 of SEQ ID NOs: 17, 18 and 19, respectively, and a variable light domain having VLCDRs 1-3 of SEQ ID NOs: 21, 22 and 23, respectively wherein one or more CDRs have 3 or fewer single amino acid substitutions, deletions or insertions.

In one instance, the method of the first aspect further comprises a step of contacting the complexes with one or more reporter molecules capable of binding to the one or more complexes. In such an instance, the detecting step (b) comprises detecting the levels of bound reporter molecules in said sample.

In one instance, the reporter molecules are as defined hereinbelow.

In one instance, step (a) comprises contacting the sample with one or more binding molecules capable of binding to reduced IL-33. Suitably in such an instance, the one or more binding molecules comprise those defined in (i). Suitably in such an instance, the one or more binding molecules consist of those defined in (i). Suitably, in such an instance, step (b) comprises detecting the levels of the reduced IL-33-binding molecule complexes.

In one instance, step (a) comprises contacting the sample with one or more binding molecules capable of binding to oxidised IL-33. Suitably in such an instance, the one or more binding molecule comprise those defined in (ii). Suitably in such an instance, the one or more binding molecule consist of those defined in (ii). Suitably, in such an instance, step (b) comprises detecting the levels of the oxidised IL-33-binding molecule complexes.

In one instance, step (a) comprises contacting the sample with one or more binding molecules capable of binding to IL-33/sST2. Suitably in such an instance, the one or more binding molecules comprise those defined in (iii). Suitably in such an instance, the one or more binding molecules consist of those defined in (iii). Suitably, in such an instance, step (b) comprises detecting the levels of the IL-33/sST2-binding molecule complexes.

In one instance, step (a) comprises contacting the sample with one or more binding molecules capable of binding to reduced IL-33 and IL-33/sST2. Suitably in such an instance, at least one of the binding molecules is as defined in (i) or (iii). Suitably in such an instance, the binding molecules comprise those defined in (i) and (iii). Suitably in such an instance, the binding molecules consist of those defined in (i) and (iii). Suitably, in such an instance, step (b) comprises detecting the levels of reduced IL-33-binding molecule complexes and IL-33/sST2-binding molecule complexes.

In one instance, step (a) comprises contacting the sample with one or more binding molecules capable of binding to oxidised IL-33 and IL-33/sST2. Suitably in such an instance, at least one of the binding molecules is as defined in (ii) or (iii). Suitably in such an instance, the binding molecules comprise those defined in (ii) and (iii). Suitably in such an instance, the binding molecules consist of those defined in (ii) and (iii). Suitably, in such an instance, step (b) comprises detecting the levels of oxidised IL-33-binding molecule complexes and IL-33/sST2-binding molecule complexes.

In one instance, step (a) comprises contacting the sample with one or more binding molecules capable of binding to oxidised IL-33 and reduced IL-33. Suitably in such an instance, at least one of the binding molecules is as defined in (i) or (ii). Suitably in such an instance, the binding molecules comprise those defined in (i) and (ii). Suitably in such an instance, the binding molecules consist of those defined in (i) and (ii). Suitably, in such an instance, step (b) comprises detecting the levels of reduced IL-33-binding molecule complexes and oxidised IL-33-binding molecule complexes.

In one instance, step (a) comprises contacting the sample with one or more binding molecules capable of binding to oxidised IL-33, reduced IL-33, and IL-33/sST2. Suitably in such an instance, at least one of the binding molecules is as defined in (i), (ii) or (iii). Suitably in such an instance, the binding molecules comprise those defined in (i), (ii) and (iii). Suitably in such an instance, the binding molecules consist of those defined in (i), (ii) and (iii). Suitably, in such an instance, step (b) comprises detecting the levels of reduced IL-33-binding molecule complexes, oxidised IL-33-binding molecule complexes, and IL-33/sST2-binding molecule complexes.

According to a second aspect of the present disclosure there is provided a method for detecting antigenically distinct forms of IL-33 in a biological sample, comprising:
(a) contacting the sample with one or more binding molecules capable of binding to reduced IL-33, oxidised IL-33, and IL-33/sST2 under conditions sufficient to form complexes; and
(b) detecting the levels of the reduced IL-33, oxidised IL-33, and IL-33/sST2 complexes in said sample.

In one instance, the binding molecules are as defined in the fourth, fifth and sixth aspects.

In one instance, the method of the second aspect further comprises a step of contacting the complexes with one or more reporter molecules capable of binding to the complexes. In such an instance, the detecting step (b) comprises detecting the levels of bound reporter molecules in said sample.

In one instance, the reporter molecules are as defined hereinbelow.

According to a third aspect of the present disclosure, there is provided an assay kit for detecting antigenically distinct forms of IL-33 in a biological sample, comprising:
(a) A binding molecule capable of binding to an antigenically distinct form of IL-33 selected from: reduced IL-33, oxidised IL-33 and/or IL-33/sST2 to form a complex;
(b) A reporter molecule capable of binding to a binding molecule complex selected from: a reduced IL-33, an oxidised IL-33 and/or IL-33/sST2 binding molecule complex.

In one instance, the binding molecules are as defined in the fourth, fifth and sixth aspects. In one instance the binding molecules may be regarded as capture molecules or probes.

In one instance, the reporter molecules are as defined hereinbelow. In a further instance, the reporter molecules many be those defined in the seventh aspects.

According to a fourth aspect of the present disclosure, there is provided a binding molecule which binds to reduced IL-33 comprising:
a. a variable heavy domain having VHCDRs 1-3 of SEQ ID NOs: 1, 2 and 3, respectively, and a variable light domain having VLCDRs 1-3 of SEQ ID NOS: 5, 6 and 7, respectively, wherein one or more CDRs have 3 or fewer single amino acid substitutions, deletions or insertions;
b. a variable heavy domain having VHCDRs 1-3 of SEQ ID NOs: 25, 26 and 27, respectively, and a variable light domain having VLCDRs 1-3 of SEQ ID NOS: 29, 30 and 31, respectively, wherein one or more CDRs have 3 or fewer single amino acid substitutions, deletions or insertions; or
c. a variable heavy domain having VHCDRs 1-3 of SEQ ID NOs: 35, 36 and 37, respectively, and a variable light domain having VLCDRs 1-3 of SEQ ID NOS:

39, 40 and 41, respectively, wherein one or more CDRs have 3 or fewer single amino acid substitutions, deletions or insertions.

According to a fifth aspect of the present disclosure, there is provided a binding molecule which binds to oxidised IL-33 comprising a variable heavy domain having VHCDRs 1-3 of SEQ ID NOs: 9, 10 and 11, respectively, and a variable light domain having VLCDRs 1-3 of SEQ 5 ID NOs: 13, 14 and 15, respectively, wherein one or more CDRs have 3 or fewer single amino acid substitutions, deletions or insertions.

According to a sixth aspect of the present disclosure, there is provided a binding molecule which binds to IL-33/sST2 comprising a variable heavy domain having VHCDRs 1-3 of: SEQ ID NOs: 17, 18 and 19, respectively, and a variable light domain having VLCDRs 1-3 of SEQ 10 ID NOs: 21, 22 and 23, respectively, wherein one or more CDRs have 3 or fewer single amino acid substitutions, deletions or insertions.

In one instance any of the binding molecules defined above may be regarded as a capture molecule or probe. Suitably each capture molecule or probe may be used with a reporter molecule, suitably as defined below. Suitably in a method for detecting antigenically distinct forms of IL-33 in a biological sample as defined in the second aspect.

In one instance, the or each binding molecule may comprise a detectable label. In one instance, the or each binding molecule is conjugated to a detectable label. Suitably therefore in one instance, the or each binding molecule herein may be used as a reporter molecule.

According to a seventh aspect of the present disclosure, there is provided a reporter molecule which binds to reduced-IL33-binding molecule complexes comprising: (a) a variable heavy domain having VHCDRs 1-3 of SEQ ID NOs: 1, 2 and 3, respectively, and a variable light domain having VLCDRs 1-3 of SEQ ID NOs: 5, 6 and 7, respectively, wherein one or more CDRs have 3 or fewer single amino acid substitutions, deletions or insertions, (b) a variable heavy domain having VHCDRs 1-3 of SEQ ID NOs: 25, 26 and 27, respectively, and a variable 25 light domain having VLCDRs 1-3 of SEQ ID NOs: 29, 30 and 31, respectively, wherein one or more CDRs have 3 or fewer single amino acid substitutions, deletions or insertions, or (b) a variable heavy domain having VHCDRs 1-3 of SEQ ID NOs: 35, 36 and 37, respectively, and a variable light domain having VLCDRs 1-3 of SEQ ID NOs: 39, 40 and 41, respectively, wherein one or more CDRs have 3 or fewer single amino acid substitutions, deletions or insertions. In one instance, the or each reporter molecule may comprise a detectable label. In one instance, the or each reporter molecule is conjugated to a detectable label.

According to an alternative seventh aspect of the present disclosure there is provided a reporter molecule which binds to oxidised-IL33-binding molecule complexes comprising a variable heavy domain having VHCDRs 1-3 of SEQ ID NOs: 9, 10 and 11, respectively, and a variable light domain having VLCDRs 1-3 of SEQ ID NOs: 13, 14 and 15, respectively, wherein one or more CDRs have 3 or fewer single amino acid substitutions, deletions or insertions. In one instance, the or each reporter molecule may comprise a detectable label. In one instance, the or each reporter molecule is conjugated to a detectable label.

According to an alternative seventh aspect of the present disclosure there is provided a reporter molecule which binds to IL-33/sST2-binding molecule complexes comprising a variable heavy domain having VHCDRs 1-3 of: SEQ ID NOs: 17, 18 and 19, respectively, and a variable light domain having VLCDRs 1-3 of SEQ ID NOs: 21, 22 and 23, respectively, wherein one or more CDRs have 3 or fewer single amino acid substitutions, deletions or insertions. In one instance, the or each reporter molecule may comprise a detectable label. In one instance, the or each reporter molecule is conjugated to a detectable label.

In one instance of the fourth to seventh aspects, the binding molecules and reporter molecules are antibodies or antigen binding fragments thereof.

In one instance, there may be provided a pair of molecules comprising a binding molecule selected from the fourth to the sixth aspect, and a reporter molecule, optionally selected from one of the seventh aspects. Suitably wherein the binding molecule is according to the fourth aspect and reporter molecule is according to one of the seventh aspects. Suitably wherein the binding molecule is according to the fifth aspect and reporter molecule is according to one of the seventh aspects. Suitably wherein the binding molecule is according to the sixth aspect and reporter molecule is according to one of the seventh aspects. Alternatively, the binding molecule may be selected from the fourth to the sixth aspect, and the reporter molecule may be selected from a commercially available reporter molecule. Suitably any commercially available reporter molecule capable of binding to the reduced-IL33-binding molecule complexes, the oxidised-IL33-binding molecule complexes, or the IL-33/sST2-binding molecule complexes may be used. Suitable such commercially available reporter molecules are described elsewhere herein. Alternatively still, the binding molecule may be selected from a commercially available binding molecule, and the reporter molecule may be selected from one of the seventh aspects. Suitably any commercially available binding molecule capable of binding to reduced-IL33, oxidised-IL33, or IL-33/sST2 may be used. Suitable such commercially available binding molecules are described elsewhere herein.

Suitably the skilled person may select appropriate pairs of binding molecules and reporter molecules to detect reduced IL-33, oxidised IL-33 or IL33/sST2, from those described in the fourth to the seventh aspects and/or commercially available molecules.

For example, in one instance, there is provided a pair of molecules comprising a binding molecule according to the sixth aspect which binds to IL-33/sST2 comprising a variable heavy domain having VHCDRs 1-3 of: SEQ ID NOs: 17, 18 and 19, respectively, and a variable light domain having VLCDRs 1-3 of SEQ ID NOs: 21, 22 and 23, respectively, wherein one or more CDRs have 3 or fewer single amino acid substitutions, deletions or insertions, and a reporter molecule capable of binding to IL-33/sST2-binding molecule complexes optionally comprising a detectable label. Suitably for use in detecting reduced IL-33/sST2 in a biological sample.

For example, in one instance, there is provided a pair of molecules comprising a binding molecule capable of binding to IL-33/sST2, and a reporter molecule comprising a variable heavy domain having VHCDRs 1-3 of: SEQ ID NOs: 17, 18 and 19, respectively, and a variable light domain having VLCDRs 1-3 of SEQ ID NOs: 21, 22 and 23, respectively, wherein one or more CDRs have 3 or fewer single amino acid substitutions, deletions or insertions optionally comprising a detectable label. Suitably also for use in detecting reduced IL-33/sST2 in a biological sample.

In one instance, the pair of molecules are for use in detecting reduced IL-33 in a biological sample. In one instance, the pair of molecules are for use in detecting oxidised IL-33 in a biological sample. In one instance, the pair of molecules are for use in detecting reduced IL-33/sST2 in a biological sample.

Suitably as described above, when used in a pair of molecules with a reporter molecule, the binding molecule may be regarded as a capture molecule or probe.

In an eighth aspect of the present disclosure, there is provided a polynucleotide encoding a VH or VL domain of any of the binding molecules of the fourth, fifth, or sixth aspects, or a polynucleotide encoding the VH or VL domain of the reporter molecule of the seventh aspect. In a ninth aspect of the present disclosure, there is provided a polynucleotide encoding any of the binding molecules of the fourth, fifth, or sixth aspects, or a polynucleotide encoding the reporter molecule of the seventh aspect.

In a tenth aspect there is provided a vector comprising one or more polynucleotides of the eighth or ninth aspects.

In an eleventh aspect there is provided a host cell comprising one or more polynucleotides of the eighth or ninth aspects or one or more vectors of the tenth aspect. In one embodiment, the host cell may comprise a first vector comprising a polynucleotide encoding the VH domain of a binding molecule of the fourth, fifth, or sixth aspects, and a second vector comprising a polynucleotide encoding the corresponding VL domain of a binding molecule of the fourth, fifth, or sixth aspects.

In a twelfth aspect there is provided a method of producing a binding molecule of the fourth, fifth, or sixth aspects, comprising: culturing a host cell of the eleventh aspect which comprises one or more polynucleotides encoding the binding molecule, under suitable conditions for the host cell to express the binding molecule, and recovering said binding molecule.

In a thirteenth aspect there is provided a binding molecule produced by the twelfth aspect.

In a fourteenth aspect there is provided a composition comprising a binding molecule of the fourth, fifth, or sixth aspects.

In a fifteenth aspect there is provided the use of a binding molecule of the fourth, fifth, or sixth aspect, or a reporter molecule of the seventh aspect, in an assay. In one embodiment, the assay is an immunoassay such as an ELISA, ECLIA, or CLIA. In one embodiment, the assay comprises a method of detecting one or more antigenically distinct forms of IL-33 in a biological sample. In one embodiment, the method is according to the first aspect.

Further features and instances of the above defined aspects are described hereinbelow in headed sections. Each section is combinable with any of the above mentioned aspects in any compatible combination.

### DESCRIPTION OF THE FIGURES

The disclosure will now be described with reference to the following figures in which:

FIG. 1A: shows MSD assays (top) and MSD S-PLEX assays® (bottom) for recombinant protein standards for reduced and oxidised IL-33. AB1070012 was used as the capture antibody and AB1070019 was used as the detection antibody.

FIG. 1B: shows MSD assays (top) and MSD S-PLEX assays® (bottom) for recombinant protein standards for reduced and oxidised IL-33. AB1070141 was used as the capture antibody and AF3625 (R&D Systems) was used as the detection antibody.

FIG. 1C: shows MSD assays (top) and MSD S-PLEX assays® (bottom) for recombinant protein standards for IL-33/sST2. AB1070008 was used as the capture antibody and MAB5232 (R&D Systems) was used as the detection antibody.

FIG. 2A: Shows examples of measurement of released endogenous forms of human IL-33 in healthy individuals and patient samples and in in vitro human epithelial cell culture systems using the assays of the disclosure. (A) Shows an example of MSD S-PLEX assays® used to detect different IL-33 forms in commercial human serum samples (n=50) from healthy individuals (n=20), asthma (severity GINA 2-3, n=14) and COPD patients (severity GOLD 2-3, n=16). Concentrations of IL-33 forms in samples were extrapolated using the relevant purified recombinant protein standard curves.

FIG. 2B Shows an example of the use of MSD assays for measurement of reduced IL-33 (n=14), oxidised IL-33 (n=4) and IL-33/sST2 (n=6) in nasal mucosal lining fluids at baseline (top) and after intranasal grass pollen allergen challenge (30 min before to 480 min after challenge) (bottom). Concentrations of IL-33 forms in samples were extrapolated using the relevant purified recombinant protein standard curves.

FIG. 2C Shows an example of the use of MSD S-PLEX assays® for measurement of reduced and oxidised IL-33 released from submerged healthy human bronchial epithelial cells (NHBE) into culture supernatants (time course of release 1-1440 min). Concentrations of IL-33 forms in samples were extrapolated using the relevant purified recombinant protein standard curves.

FIG. 2D Shows an example of use of MSD assays for measurement of reduced and oxidised IL-33 released from human epithelial air-liquid interface (ALI) into culture supernatants (n=7 donors, after 24 h). Concentrations of IL-33 forms in samples were extrapolated using the relevant purified recombinant protein standard curves.

FIG. 3: shows the two ACCORD recruitment periods during the pandemic.

FIG. 4: Shows the measurement of IL33-sST2 in patients during the ACCORD Phase 2a study using the assay of the disclosure. The numbers of patients treated with SoC versus SoC combined with tozorakimab treatment that died or had respiratory failure at day 29 of the study are shown (a) overall, (b) grouped according to low IL-33/sST2 baseline levels as measured by the assay, or (c) grouped according to high IL-33/sST2 baseline levels as measured by the assay.

FIG. 5 shows that both AB1070012 and AB1070069 show equivalent performance in the MSD assay format for detecting reduced IL-33 when paired with AB1070019

### DETAILED DESCRIPTION

### Definitions

‘IL-33’ protein as employed herein refers to interleukin 33, in particular a mammalian interleukin 33 protein, for example human protein deposited with UniProt number 095760. However, given the present inventors findings, it clear that this entity is not a single species but instead exists as reduced and oxidized forms. The terms “IL-33” and “IL-33 polypeptide” are used interchangeably. In certain instances, IL-33 is full length. In another instance, IL-33 is mature, truncated IL-33 (amino acids 112-270). Recent studies suggest full length IL-33 is active (Cayrol and Girard, Proc Natl Acad Sci USA 106 (22): 9021-6 (2009); Hayakawa et al., Biochem Biophys Res Commun. 387 (1): 218-22 (2009); Talabot-Ayer et al, J Biol Chem. 284 (29): 19420-6 (2009)). However, N-terminally processed or truncated IL-33 including but not limited to aa 72-270, 79-270, 95-270, 99-270, 107-270, 109-270, 111-270, 112-270 may have enhanced activity (Lefrancais 2012, 2014). In another instance, IL-33 may include a full length IL-33, a fragment thereof, or an IL-33 mutant or variant polypeptide, wherein the fragment of IL-33 or IL-33 variant polypeptide retains some or all functional properties of active IL-33.

‘Oxidized IL-33’ or ‘oxIL-33’ as employed herein refers to the form of the IL-33 that binds to RAGE, and triggers RAGE-EGFR mediated signalling. Oxidised IL-33 is a protein visible as a distinct band, for example by western blot analysis under non-reducing conditions, in particular with a mass 4 Da less than the corresponding reduced from. In particular, it refers to a protein with one or two disulphide bonds between the cysteines independently selected from cysteines 208, 227, 232 and 259. In one instance, oxidized IL-33 shows no binding to ST2.

‘Reduced IL-33’ or ‘redIL-33’ as employed herein refers to the form of the IL-33 that binds to ST2 and triggers ST2 mediated signalling. In particular cysteines 208, 227, 232 and 259 of the reduced form are not disulfide bonded. In one instance, reduced IL-33 shows no binding to RAGE.

‘IL-33/sST2’ as employed herein refers to the form of reduced IL-33 that is bound to soluble ST2 receptors, this form may also be referred to as a complex.

‘Antigenically distinct forms of IL-33’ as employed herein refers to any form of IL-33 which can act as an antigen and be discriminated by an antibody or binding fragment thereof, typically in the context of the present disclosure this means oxidised IL-33, reduced IL-33 and reduced IL-33/sST2 complexes.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al, J. Biol. Chem. 260:2605-2608 (1985); and Rossolini et al, Mol. Cell. Probes 8:91-98 (1994)).

The term “conservative sequence modifications” refers to amino acid modifications that do not significantly affect or alter the binding characteristics of the antibody or antibody fragment containing the amino acid sequence. Such conservative modifications include amino acid substitutions, additions, and deletions. Modifications can be introduced into an antibody or antibody fragment of the invention by standard techniques known in the art, such as site-directed mutagenesis and PCR-mediated mutagenesis.

It is to be noted that the term “a” or “an” entity refers to one or more of that entity; for example, “an anti-IL-33 antibody” is understood to represent one or more anti-IL-33 antibodies. As such, the terms “a” (or “an”), “one or more,” and “at least one” can be used interchangeably herein.

By “subject” or “individual” or “animal” or “patient” or “mammal,” is meant any subject, particularly a mammalian subject, for whom diagnosis, prognosis, or therapy is desired, except where the subject is defined as a ‘healthy subject’. Mammalian subjects include humans; domestic animals; farm animals; such as dogs, cats, guinea pigs, rabbits, rats, mice, horses, cattle, cows, and so on.

### Sample

The present disclosure relates to assaying a biological sample by measuring the level of antigenically distinct forms of IL-33 present in the sample.

Suitably, the sample is a biological sample. Suitably, the sample is a body fluid sample. Alternatively, suitable samples may include tissue samples, such as biopsies.

A suitable body fluid sample may include: a blood sample (for example, a whole blood sample, a blood plasma sample, or a serum sample); or a mucosal lining fluid sample (for example mucosal lining fluid from an epithelium); or a lavage sample (for example a lung or bronchoalveolar lavage sample); or a supernatant sample (for example from a culture of epithelium cells).

Suitably a supernatant sample may be an epithelial cell supernatant sample. Suitably from a bronchial epithelial culture. Suitably the supernatant may be from the cells or from the air-liquid interface of the culture.

Suitably the different antigenically distinct forms of IL-33 may be present in different body fluids. Suitably therefore the different antigenically distinct forms of IL-33 may be measured in different body fluid samples.

Suitably oxidised IL-33 is measured in a mucosal lining fluid sample or a lavage sample.

Suitably reduced IL-33 is measured in a mucosal lining fluid sample or a lavage sample.

Suitably IL-33/sST2 complexes are measured in a serum sample.

Suitably the mucosal fluid lining samples or lavage samples contain biological cells, suitably these samples contain epithelium cells.

Suitably the methods of the disclosure may further comprise a step of obtaining a biological sample. Suitably obtaining a biological sample from a subject. Suitably this step takes place before the contacting step (a). Alternatively, the sample may have been previously been obtained.

Suitably the subject may be a healthy subject or may have a disease. Suitably therefore the subject may be a patient. Suitably therefore, the methods of the disclosure may carried out on biological samples obtained from patients with a disease. Suitably therefore methods of the disclosure may further comprise a step of obtaining a biological sample from a patient.

Suitably a blood sample may be taken by a blood draw from the subject.

Suitably a mucosal lining fluid sample may be taken by a nasal swab from the subject.

Suitably a lavage sample may be taken by a lung wash from the subject.

Suitably a supernatant sample may be taken by culturing cells from the subject. Suitably by culturing epithelium cells isolated from the epithelium of a subject. Suitably isolated from the respiratory epithelium of a subject.

Suitably, the sample is representative of cytokine levels in the subject in respect of whom the method is being practised. Suitably, the sample is representative of interleukin levels in a subject in respect of whom the method is being practised. Suitably, the sample is representative of IL-33 levels in a subject in respect of whom the method is being practised.

Suitably the sample is a mucosal lining fluid sample from the respiratory tract, suitably from the epithelium of the respiratory tract. Suitably this may be isolated from the upper or lower respiratory tract, suitably from the epithelium of the upper or lower respiratory tract. Suitably it is isolated from the upper respiratory tract, suitably from the epithelium of the upper respiratory tract. Suitably, the sample is nasal mucosal lining fluid, suitably nasal mucosal lining fluid isolated from the epithelium of the nasal canals.

Nasal mucosal lining fluid samples are particularly suitable for use in the methods of the disclosure. The use of such samples is advantageous, since they are readily accessible. Additionally obtaining the sample is associated with much less risk and discomfort than is the case for samples such as lung tissue samples.

Samples may be processed for the enrichment of antigenically distinct forms of IL-33. Suitable techniques for such enrichment may be determined with reference to the nature of the sample. Generally, examples of suitable techniques (such as techniques for the isolation of biological molecules such as cytokines from a sample) will be well known to those skilled in the art.

### Subject

The methods of the disclosure may be are practiced in respect of a sample obtained from a subject.

Suitably the subject may be a human. The subject may be undergoing medical care, or may be requesting medical care. Suitably the subject is male or female. Suitably the subject is an adult or a child.

As explained above, the subject may be healthy or may have a disease. Suitably the subject may be a patient.

Suitably in the methods of the present disclosure, a suitable subject may be one suspected of having abnormal levels of antigenically distinct forms of IL-33. Suitably, the subject may be one believed to have a physiological condition or disease which involves abnormal levels of antigenically distinct forms of IL-33, or a subject where a prognosis or diagnosis of a condition or disease depends on the level of one or more antigenically distinct forms of IL-33. Suitably, abnormal levels of antigenically distinct forms of IL-33 may be a result or a cause of the disease or condition.

Suitably the subject may display symptoms of such conditions or diseases.

Alternatively, a suitable subject in the context of the methods of the present disclosure may be one believed to be at risk of developing such a condition or disease. For example, such a subject may have been in contact with an individual suffering from such diseases or condition, may suffer from a related condition, or may satisfy risk factors associated with said conditions like smoking, old age, allergy etc.

### Contacting

The methods of the disclosure involve a step of contacting a biological sample with one or more binding molecules capable of specifically binding to one or more antigenically distinct forms of IL-33 selected from: reduced IL-33, oxidised IL-33, and/or IL-33/sST2 under conditions sufficient to form complexes.

Suitably the methods of the disclosure involve a step of contacting a biological sample with one or more binding molecules capable of specifically binding to one or more antigenically distinct forms of IL-33 selected from: reduced IL-33, oxidised IL-33, and/or IL-33/sST2.

Suitably by ‘specifically binding’ it is meant that each binding molecule is capable of binding to a specific form of IL-33.

Suitably, the methods of the disclosure may also comprise a step of contacting the complexes with one or more reporter molecules capable of binding to the one or more complexes. In such an instance, the detecting step (b) comprises detecting the levels of bound reporter molecules in said sample. In such an instance, suitably the methods further comprise a step of contacting the biological sample with one or more reporter molecules capable of binding to the one or more complexes therein. Suitably for a sufficient time to allow the reporter molecules to bind to the one or more complexes.

Suitably, the methods of the disclosure may also comprise a step of contacting the complexes with one or more reporter molecules capable of specifically binding to the one or more complexes. Suitably contacting the complexes with one or more reporter molecules may comprise contacting the biological sample with one or more reporter molecules.

Suitably by ‘specifically binding’ it is meant that each reporter molecule is capable of binding to a specific form of IL-33-binding molecule complex.

Suitable binding molecules and reporter molecules are defined elsewhere herein.

Optionally the methods of the disclosure may comprise a further step prior to contacting the sample with one or more binding molecules capable of specifically binding to one or more antigenically distinct forms of IL-33, the further step comprising immobilizing the antigenically distinct isoforms of IL-33 by contacting the biological sample with a solid surface under suitable conditions for the antigenically distinct isoforms of IL-33 to bind to the solid surface, suitably by adsorption or covalent bonding. Suitably the solid surface may comprise a plate such as a microtitre plate or multiwell plate, a well, a slide such as a glass slide, paper such as filter paper, a cuvette, or the like. Suitably the solid surface is comprised in the container in which the method will be performed. Suitably therefore a solid surface comprising the antigenically distinct isoforms of IL-33 is produced. Suitably, in such instances, the binding molecules are then contacted with the solid surface comprising the antigenically distinct isoforms of IL-33 for a sufficient time to form complexes.

Suitably the binding molecules are contacted with the biological sample for a sufficient time to form complexes. Suitably the reporter molecules are contacted with the biological sample for a sufficient time to bind to the one or more complexes.

Suitably this time may be termed an incubation time. Suitably, incubation times will vary depending on the binding molecules used and the reporter molecules used, as well as the biological sample.

Suitably a sufficient incubation time may be between 5 minutes and 180 minutes, suitably between 10 minutes and 150 minutes, suitably between 20 minutes and 120 minutes, suitably between 30 minutes and 100 minutes, suitably between 45 minutes and 90 minutes, suitably around 60 minutes.

Suitably the one or more binding molecules are contacted with the biological sample for around 120 minutes, 100 minutes, 80 minutes, 60 minutes, 40 minutes, 30 minutes, 25 minutes, 20 minutes, 15 minutes, 10 minutes, 9 minutes, 8 minutes, 7 minutes, 6 minutes or 5 minutes. Suitably the one or more binding molecules are contacted with the biological sample for around 9 minutes.

Suitably the one or more reporter molecules are contacted with the complexes/biological sample for around 120 minutes, 100 minutes, 80 minutes, 60 minutes, 40 minutes, 30 minutes, 25 minutes, 20 minutes, 15 minutes, 10 minutes, 9 minutes, 8 minutes, 7 minutes, 6 minutes or 5 minutes. Suitably the one or more reporter molecules are contacted with the complexes/biological sample are contacted with the biological sample for around 9 minutes.

Suitably contacting the biological sample with the binding molecules or reporter molecules comprises bringing the binding molecules or reporter molecules and the biological sample together such that binding can occur.

Suitably the biological sample may be contacted with the binding molecules or reporter molecules in any suitable way using known assay techniques.

The sample can be brought in contact with the one or more binding molecules by using a solid phase support or carrier. Suitably, either the sample or binding molecules may be immobilised onto the support or carrier.

Suitably, the support may then be washed. Suitably the support is washed with a suitable buffer. Suitably the methods of the disclosure may comprise one or more steps of washing. Suitably washing takes place after the addition of the one or more binding molecules and before addition of the one or more reporter molecules. Suitably after the addition of the one or more reporter molecules and before the determining step.

Suitably at least one washing step occurs after step (a), and suitably before step (b).

Suitably, the support may be contacted with the one or more reporter molecules after step (a). Suitably therefore, the step of contacting the complexes with one or more reporter molecules capable of binding to the one or more complexes may comprise contacting the complexes immobilised on a support with one or more reporter molecules capable of binding to the one or more complexes. Suitably the support may then be washed a second time to remove unbound reporter molecules. Suitably therefore a second washing step may occur after contacting the complexes with the one or more reporter molecules.

Optionally the binding molecules or reporter molecules may comprise a detectable label or they may be subsequently labelled. Suitable labels are discussed elsewhere herein. The amount of bound label, which may be bound on the solid support, may then be detected by conventional means, suitably as recited in step (b) of the methods of the disclosure.

Suitably in some embodiments wherein the assay is an immunoassay, the binding molecules may be biotinylated. Suitably in some embodiments wherein the assay is an immunoassay, the reporter molecules may be labelled with a chemiluminescent compound.

By “solid phase support or carrier” it is intended to mean any support capable of binding an antigen or an antibody. Well-known supports or carriers include glass, polystyrene, polypropylene, polyethylene, dextran, nylon, nitrocellulose, amylases, natural and modified celluloses, polyacrylamides, and magnetite. The nature of the carrier can be either soluble to some extent or insoluble for the purposes of the present disclosure. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod.

Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

### Detecting

The methods of the disclosure involve a step of detecting the levels of the one or more reduced IL-33, oxidised IL-33, and/or IL-33/sST2 complexes in a biological sample.

Suitably detecting the levels of the one or more reduced IL-33, oxidised IL-33, and/or IL-33/sST2 complexes refers to detecting the levels of reduced IL-33-binding molecule complexes and/or oxidised IL-33-binding molecule complexes and/or IL-33/sST2-binding molecule complexes. Suitably such complexes are formed in step (a) from the one or more binding molecules binding specifically to one or more of reduced IL-33, oxidised IL-33, and/or IL-33/sST2 in the biological sample.

Suitably the complexes detected in step (b) correspond to the binding molecules used in the method. Suitably therefore if the method is carried using only oxidised IL-33 binding molecules, then only oxidised IL-33 complexes are detected in step (b).

Suitably, detection of the one or more reduced IL-33, oxidised IL-33, and/or IL-33/sST2 complexes in a sample may be performed by any suitable assay technique known in the art.

Suitably, as mentioned above, detecting the one or more reduced IL-33, oxidised IL-33, and/or IL-33/sST2 complexes in a sample may comprise detecting the levels of one or more bound reporter molecules in said sample. Suitably, detecting the levels of one or more reporter molecules bound to the one or more reduced IL-33, oxidised IL-33, and/or IL-33/sST2 complexes in the sample.

Suitably the reporter molecules are detected by detection of a detectable label associated with each reporter molecule. Suitably the detectable label may be part of the reporter molecule or may be added separately, suitably after the reporter molecule has bound to the one or more reduced IL-33, oxidised IL-33, and/or IL-33/sST2 complexes in the sample.

Alternatively, detecting the one or more reduced IL-33, oxidised IL-33, and/or IL-33/sST2 complexes in a sample may comprise detecting the levels of the one or more bound binding molecules in said sample. In such an instance, suitably the binding molecules are directly detected by detection of a detectable label associated with each binding molecule. Suitably, in such an instance, no reporter molecules are required. Suitably the detectable label may be part of the binding molecule or may be added separately, suitably after the binding molecule has bound to the one or more reduced IL-33, oxidised IL-33, and/or IL-33/sST2 complexes in the sample.

Suitable detectable labels are discussed elsewhere herein.

Suitably therefore the methods of the disclosure may further comprise a step of adding a detectable label to the sample. Suitably, step (b) may comprise detecting the detectable label associated with the one or more reporter molecules.

Alternatively, step (b) may comprise detecting the detectable label associated with the one or more binding molecules.

In one instance, the reporter molecules comprise a detectable label conjugated thereto. In such an instance, the reporter molecules are detected by detection of the conjugated detectable label.

In one instance, the binding molecules comprise a detectable label conjugated thereto. In such an instance, the binding molecules are detected by detection of the conjugated detectable label.

Suitably the assay used is highly sensitive and therefore the methods of the disclosure are highly sensitive. Suitably the methods of the disclosure are capable of detecting antigenically distinct forms of IL-33 in a sample at a concentration of microgram/ml or lower, nanogram/ml or lower, picogram/ml or lower, femtogram/ml or lower.

Suitably the lower limit of detection is a concentration of microgram/ml or lower, nanogram/ml or lower, picogram/ml or lower, femtogram/ml or lower. Suitably the lower limit of detection is a concentration of femtogram/ml.

In one instance, the method is capable of detecting antigenically distinct forms of IL-33 in a sample at a concentration of at least femtogram/ml.

### Assay

Suitably the methods of the disclosure may comprise detecting one or more antigenically distinct forms of IL-33 in a biological sample in a single assay.

Alternatively, the methods of the disclosure may comprise detecting one or more antigenically distinct forms of IL-33 in a biological samples in more than one assay. Suitably, therefore the methods of the disclosure may also comprise more than one biological sample. Suitably, therefore the methods of the disclosure may comprise more than one step within step (a) and more than one step within step (b).

Suitably, the methods of the disclosure may comprise separately contacting one or more biological samples with one or more binding molecules capable of binding reduced IL-33, oxidised IL-33, and/or IL-33/sST2.

Suitably step (a) of the methods of the disclosure may comprise: (ai) contacting a first sample with a binding molecule capable of binding to oxidised IL-33; (aii) contacting a second sample with a binding molecule capable of binding to reduced IL-33; and/or (aiii) contacting a third sample with a binding molecule capable of binding to IL-33/sST2. Suitably wherein the first second and third samples may be the same sample or different samples.

Suitably, the methods of the disclosure may also comprise separately detecting the levels of one or more reduced IL-33, oxidised IL-33, and/or IL-33/sST2 complexes in one or more samples.

Suitably step (b) of the methods of the disclosure may comprise: (bi) detecting the level of oxidised IL-33-binding molecule complexes in a first sample; (bii) detecting the level of reduced IL-33-binding molecule complexes in a second sample; and/or (biii) detecting the level of IL-33/sST2-binding molecule complexes in a third sample. Suitably wherein the first, second or third samples may be the same sample or different samples.

As noted above, the different antigenically distinct forms or IL-33 may be present in the same or different samples, therefore suitably the different antigenically distinct forms of IL-33 may require different assays for different samples. Suitably therefore the methods may comprise one or more assays.

Suitably step (a) of the methods of the disclosure may comprise: (ai) contacting a mucosal lining fluid or lung lavage sample with an oxidised IL-33 binding molecule; (aii) contacting a mucosal lining fluid or lung lavage sample with a reduced IL-33 binding molecule; and/or (aiii) contacting a serum sample with an IL-33/sST2 binding molecule.

Suitably any of the antigenically distinct forms of IL-33 may be detected in the same sample or different samples in any combination.

For example, suitably the methods of the disclosure may comprise: (bi) detecting the level oxidised IL-33-binding molecule complexes and level of reduced IL-33-binding molecule complexes in a first sample; and/or (bii) detecting the level of IL-33/sST2-binding molecule complexes in a second sample. For example, suitably the methods of the disclosure may comprise (bi) detecting the level oxidised IL-33-binding molecule complexes and level of reduced IL-33-binding molecule complexes in a mucosal lining fluid or lung lavage sample; and/or (bii) detecting the level of IL-33/sST2-binding molecule complexes in a serum sample.

Prior to step (a), the methods of the disclosure may further comprise a step of isolating the one or more antigenically distinct forms of IL-33 from a biological sample. Suitably the isolation step may comprise isolation by fractionation of the sample. Suitable isolation techniques are known in the art and may comprise: chromatography, centrifugation, affinity purification, filtration and the like. Suitably the isolation step may comprise immobilizing the one or more antigenically distinct forms of IL-33 onto a solid surface, as explained elsewhere herein.

Examples of suitable samples are described elsewhere in the present specification. It will be appreciated that the nature of a sample may then determine the nature of assay techniques that may be used in practicing a method of the disclosure.

The various forms of IL-33 are all proteins. Suitably therefore the assay technique is a protein assay technique.

Protein levels can be determined by various assay techniques such as S-plex, ELISA, ECLIA, CLIA, radioimmunoassay, immunoprecipitation, Western blot and mass spectrometry.

Suitably the level of one or more antigenically distinct forms of IL-33 is determined by an immunoassay.

Immunoassays typically require capture reagents, such as antibodies, to capture the relevant analyte, and optionally probe reagents, to detect the relevant analyte. Suitable immunoassay techniques are: ELISA (enzyme linked immunosorbent assay), ECLIA (enzyme electrochemiluminescent immunoassay) such as Elecsys® from Roche, S-plex, CLIA (chemiluminescence immunoassay), western blotting, immunocytochemistry, immunoprecipitation, affinity chromotography, Bio-Layer Interferometry, (Octet, ForteBio) and biochemical assays such as Dissociation-Enhanced Lanthanide Fluorescent Immunoassays (DELFIA®, Perkin Elmer), Forster resonance energy transfer (FRET) assays (e.g. homogeneous time resolved fluorescence (HTRF®, Cis Biointernational), and radioimmuno/radioligand binding assays.

Western blot analysis generally comprises preparing protein samples, electrophoresis of the protein samples in a polyacrylamide gel (e.g., 8-20 SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 1251) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding Western blot protocols see, e.g., Ausubel et al., eds, (1994) Current Protocols in Molecular Biology (John Wiley & Sons, Inc., NY) Vol. 1 at 10.8.1.

ELISAs comprise preparing antigen, coating the well of a 96-well microtiter plate with the antigen, adding an antibody conjugated to a detectable compound such as an enzymatic substrate (e.g., horseradish peroxidase or alkaline phosphatase) to the well and incubating for a period of time, and detecting the presence of the antigen. In ELISAs the antibody does not have to be conjugated to a detectable compound; instead, a second antibody (which recognizes the first antibody) conjugated to a detectable compound may be added to the well. The first antibody and the second antibody both bind to the antigen and form a ‘sandwich’. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, (1994) Current Protocols in Molecular Biology (John Wiley & Sons, Inc., NY) Vol. 1 at 13.2.1.

Suitably the levels of one or more antigenically distinct forms of IL-33 are determined by immunoassay. Suitably by using a modified ELISA called an S-plex assay. S-plex assays are available from Meso Scale Diagnostics LLC with suitable instructions for use.

Alternatively the levels of one or more antigenically distinct forms of IL-33 are determined by ECLIA. Suitably an ECLIA assay comprises contacting a sample containing an antigen with a first antibody specific for the antigen conjugated to biotin and incubating for a period of time to allow the first antibody to bind to the antigen in the sample. A second antibody specific for the antigen but conjugated to a labelling molecule is then added, typically the labelling molecule is a ruthenium compound, suitably ruthenium bipyridine. The first antibody and the second antibody both bind to the antigen and form a ‘sandwich’. In some cases, the first and second antibodies may be added at the same time. Then, simultaneously or separately, a streptavidin coated bead or microparticle is added to the sample, which binds to the biotin of the first antibody and forms antibody complexes. Suitably the beads or microparticles are magnetic. The sample is then passed over an electrode, to which the antibody complexes bind via the streptavidin beads or microparticles. Unbound antibody is washed away, with a wash buffer such as ProCell, before a reactant is added and an electric current is passed through the electrode to induce chemiluminescence of the labelling molecule. Suitably the reactant may be an amine, such as tripropylamine, or dibutyl ethanolamine. The level of chemiluminescence can be measured by a photomultiplier to indicate the amount of bound complexes and hence the amount of antigen in the sample. An example of such ECLIA immunoassays are the Elecsys® assays available from Roche, which may suitably be carried out on a Cobas e immunoassay analyzer.

In one embodiment of the present disclosure there is provided a method for detecting one or more antigenically distinct forms of IL-33 in a biological sample, comprising carrying out an ECLIA assay, suitably using one or more of the binding molecules and/or reporter molecules described herein.

In one embodiment of the present disclosure therefore, there is provided a method for detecting one or more antigenically distinct forms of IL-33 in a biological sample, comprising:
(a) contacting a sample with one or more binding molecules each capable of binding specifically to an antigenically distinct form of IL-33 selected from: reduced IL-33, oxidised IL-33, and/or IL-33/sST2, under conditions sufficient to form complexes, wherein the binding molecules each comprise a tag; (b) contacting the sample with one or more reporter molecules each capable of binding to the complexes produced in (a), under conditions sufficient to form sandwich complexes, wherein the reporter molecules each comprise a label;
(c) contacting the sample with one or more capture partners each capable of binding to the tag comprised in each binding molecule under conditions sufficient to form captured sandwich complexes;
(d) detecting the amount of captured sandwich complexes using the labels.

Suitably the binding molecules and reporter molecules are as defined elsewhere herein.

Suitably the tag is biotin. Suitably therefore the capture partner is streptavidin. However, suitably other known tag and capture partners may be used. Suitably the capture molecule may be attached to a support, suitably to a solid support, suitably to a bead or microparticle. Suitably therefore, step (c) comprises contacting the sample with one or more capture beads comprising streptavidin, each capable of binding to the biotin tag comprised in each binding molecule. Suitably therefore step (c) produces beads comprising one or more sandwich complexes.

Suitably the beads are formed of a magnetic material, suitably a magnetic metal such as iron, nickel or cobalt. Suitably therefore step (c) comprises contacting the sample with one or more magnetic capture beads comprising streptavidin, suitably coated in streptavidin, each capable of binding to the biotin tag comprised in each binding molecule.

Suitably the label is a fluorescent or luminescent, or a chemiluminescent label as identified hereinabove. Suitably the label is a chemiluminescent label, suitably the label is a ruthenium compound, suitably ruthenium pyridine.

Suitably detecting the captured sandwich complexes comprises detecting the label, suitably detecting the level of luminescence produced by the label. Suitably the level of luminescence corresponds to the level of antigen present in the sample, suitably to the level of antigenically distinct form of IL-33 selected from: reduced IL-33, oxidised IL-33, and/or IL-33/sST2 in the sample.

Suitably chemiluminescent labels require activation in order to luminesce and be detected. Suitably therefore the method comprises a step of activating the labels attached to each reporter molecule within the captured sandwich complexes. Suitably prior to the detection step. Suitably the step of activation comprises contacting the label with a stimulus, wherein the stimulus is capable of activating the labels. Suitable stimulus include chemical reactants and/or electric current/voltage. Suitably, for a chemiluminescent label, the activating step comprises both a reactant and an electric current/voltage. Suitably in proximity. Suitably therefore the method may comprise a step of contacting the captured sandwich complexes with a reactant. Suitable reactants may be electron donors, such as amine compounds, for example tripropylamine, or dibutyl ethanolamine. Suitably the step may further comprise exposing the captured sandwich complexes to an electric current/voltage, suitably at the same time as contacting with the reactant. Suitably therefore the method comprises a step of contacting the captured sandwich complexes with a reactant and passing a current through the electrode to activate the chemiluminescent label on each reporter molecule.

Suitably to ensure that the chemiluminescent label is activated, the method may comprise immobilising the captured sandwich complexes. Suitably immobilising them on a surface. In one embodiment, this may be achieved by using magnetic beads, as explained above in combination with a metal surface. Suitably the method may comprise a step of binding or immobilising the captured sandwich complexes to a magnetic surface, suitably via the magnetic beads. Suitably the surface is also capable of delivering an electric current/voltage. Suitably therefore the surface is an electrode, suitably a magnetic electrode. In one embodiment therefore, the method comprises step of a immobilising the captured sandwich complexes to a magnetic electrode, suitably via the magnetic beads of the captured sandwich complexes. Suitably this step occurs prior to the step of activating the chemiluminescent labels. Suitably prior to contacting the captured sandwich complexes with a reactant and passing a current through the electrode.

Suitably once activated the label on each reporter molecules of each captured sandwich complex can be detected. Suitably in the case of a chemiluminescent label, its luminescence can be detected. Suitably detecting such luminescence may be carried out using any known technique, for example by using a photomultiplier. Suitably the level of luminescence detected corresponds to the amount of captured sandwich complexes, which suitably corresponds to the level of antigen present in the sample. In the context of the present invention, suitably the level of luminescence corresponds to the level of reduced IL-33, oxidised IL-33, and/or IL-33/sST2 in the sample.

In one embodiment of the present disclosure therefore, there is provided a method for detecting one or more antigenically distinct forms of IL-33 in a biological sample, comprising:
(a) contacting a sample with one or more binding molecules each capable of binding specifically to an antigenically distinct form of IL-33 selected from: reduced IL-33, oxidised IL-33, and/or IL-33/sST2, under conditions sufficient to form complexes, wherein the binding molecules each comprise a biotin tag;
(b) contacting the sample with one or more reporter molecules each capable of binding to the complexes produced in (a), under conditions sufficient to form sandwich complexes, wherein the reporter molecules each comprise a chemiluminescent ruthenium label;
(c) contacting the sample with one or more magnetic capture beads coated in streptavidin, each capable of binding to the biotin tag comprised in each binding molecule, under conditions sufficient to form captured sandwich complexes;
(d) immobilising the captured sandwich complexes on a magnetic electrode via the magnetic capture beads;
(e) contacting the captured sandwich complexes with a reactant and passing a current through the electrode to activate the chemiluminescent label on each reporter molecule;
(d) detecting the level of chemiluminescence thereby detecting the one or more antigenically distinct forms of IL-33 in the sample.

Immunoassays for measuring antigenically distinct forms of IL-33 will typically comprise a step of incubating a sample, in the presence of one or more detectably labelled molecules capable of specifically binding to each of the antigenically distinct forms of IL-33 or peptide fragments thereof of interest, and a step of detecting the bound molecules by any of a number of techniques well known in the art. The methods of the disclosure follow this general format. Suitable binding molecules, labels and methods of detection are described elsewhere herein.

### Binding and Reporter Molecules

The present disclosure relates to the use of binding molecules and reporter molecules for detection of antigenically distinct forms of IL-33 in biological samples.

Whilst many techniques are known in the art for the measurement and detection of proteins such as IL-33 in samples, suitably the methods of the disclosure are carried out by immunoassay as explained above.

Suitably an immunoassay comprises the use of one or more binding molecules capable of binding to one or more antigenically distinct forms of IL-33 to form one or more complexes; and one or more reporter molecules capable of binding to the one or more complexes which are formed.

A “binding molecule” of the present disclosure refers in its broadest sense to a molecule that specifically binds an antigenic determinant. Suitably, the one or more binding molecules specifically bind to IL-33, in particular reduced IL-33, oxidised IL-33 or IL-33/sST2. In some instances, the binding molecules of the present disclosure may be regarded as capture molecules or probes.

A “reporter molecule” of the present disclosure refers in its broadest sense to a molecule that specifically binds an antigenic determinant and is capable of indicating as such. Suitably, the one or more reporter molecules specifically bind to complexes of IL-33 with a binding molecule, in particular reduced IL-33-binding molecule, oxidised IL-33-binding molecule or IL-33/sST2-binding molecule complexes.

Alternatively, the reporter molecule and binding molecule are the same entity. In such instances, the binding molecule binds directly to the antigenic determinant (e.g. reduced IL-33, oxidised IL-33 or IL-33/sST2) and is capable of indicating as such.

Suitably the molecules described herein, suitably the antibody and antigen binding fragments described herein, are each capable of being used as a binding molecule or a reporter molecule. Suitably as described in the fourth to the seventh aspects.

Suitably the one or more reporter molecules are capable of indicating that they are present and/or bound. Suitably the one or more reporter molecules are labelled. Suitably the one or more reporter molecules are detectably labelled.

Alternatively the one or more binding molecules are capable of indicating they are present and/or bound. Suitably, in such an instance, the one or more binding molecules are labelled. Suitably the one or more binding molecules are detectably labelled.

Suitable detectable labels are known in the art and may comprise a radiolabel, a fluorescent label, an enzyme, a chromophore. Suitably the label may only be detectable upon stimulation. Suitable sources of stimulation will vary depending on the label used, for example in the use of an enzyme label the source of stimulation may be a substrate, in the use of a chromophore label the source of stimulation may be radiation of a particular wavelength. Such sources of stimulation may be regarded as stimulation agents.

Suitable fluorescent labels may include: rhodamine, fluorescein, Cy5, ruthenium compounds such as pyridine complexes, diimine complexes, and phosphorescent porphyrin dyes.

Suitable enzyme labels may include: peroxidase, glucose oxidase, alkaline phosphatase (AP), β-galactosidase, catalase or luciferase. The substrates to be used with the specific enzymes are generally chosen for the production, upon hydrolysis by the corresponding enzyme, of a detectable colour change. For example, p-nitrophenyl phosphate is suitable for use with alkaline phosphatase conjugates; for peroxidase conjugates, 1,2-phenylenediamine or toluidine are commonly used. Suitably fluorogenic substrates may be used, which yield a fluorescent product.

Suitably in some embodiments wherein the assay is an immunoassay, the binding molecules may be biotinylated. Suitably in some embodiments wherein the assay is an immunoassay, the reporter molecules may be labelled with a chemiluminescent compound, suitably a ruthenium compound, suitably ruthenium bipyridine.

Therefore in a further aspect of the invention, there may be provided a binding molecule according to any of the fourth to the sixth aspects, wherein the binding molecule is biotinylated.

Therefore in a further aspect of the invention, there may be provided a reporter molecule according to the seventh aspect, wherein the reporter molecule is ruthenylated.

Alternatively, the binding molecules or reporter molecules may not be labelled. In such an instance, the binding molecules or reporter molecules may be capable of being bound by a label, suitably a detectable label. Suitably the detectable label is capable of binding the binding molecules or reporter molecule and indicating as such. Suitably the detectable label is specific to a corresponding binding molecule or reporter molecule.

Suitably the methods of the disclosure may make use of an oxidised IL-33 complex detectable label, and/or a reduced IL-33 complex detectable label and/or an IL-33/sST2 complex detectable label. Suitably such one or more detectable labels may be added to the assay methods of the disclosure after the one or more reporter molecules. Suitably the detection step (b) of the methods may comprise detecting one or more bound detectable labels.

Alternatively, the methods of the disclosure may make use of an oxidised IL-33 detectable label, and/or a reduced IL-33 detectable label and/or an IL-33/sST2 detectable label. Suitably such one or more detectable labels may be added to the assay methods of the disclosure after the one or more binding molecules. Suitably the detection step (b) of the methods may comprise detecting one or more bound detectable labels.

In one instance, the one or more reporter molecules are detectably labelled. In one instance, the one or more reporter molecules comprise a detectable label conjugated thereto. In one instance, the one or more reporter molecules are sulfo-tagged.

Suitably, the methods comprise the use of a combination of binding molecules capable of binding to several antigenically distinct forms of IL-33 to form several complexes; and a combination of corresponding reporter molecules capable of binding to the several complexes.

Suitably, the methods comprise the use of a combination of binding molecules capable of binding to oxidised IL-33, reduced IL-33, and/or IL-33/sST2 to form several complexes; and a combination of reporter molecules capable of binding to the several complexes.

Suitably, the methods comprise the use of a binding molecule capable of binding to oxidised IL-33, to form a complex; and a reporter molecule capable of binding to the oxidised IL-33-binding molecule complex.

Suitably, the methods comprise the use of a binding molecule capable of binding to reduced IL-33, to form a complex; and a reporter molecule capable of binding to the oxidised IL-33-binding molecule complex.

Suitably, the methods comprise the use of a binding molecule capable of binding to IL-33/sST2, to form a complex; and a reporter molecule capable of binding to the IL-33/sST2-binding molecule complex.

Suitably, each binding molecule has a corresponding reporter molecule. Suitably each reporter molecule may comprise a corresponding detectable label.

Suitably the reporter molecules and the binding molecules are paired. For example, when a binding molecule capable of binding to oxidised IL-33 is used, then a reporter molecule capable of binding to an oxidised IL-33-binding molecule complex is used.

Suitably, the one or more binding molecules or reporter molecules may be selected from: an antibody, an antigen-binding fragment thereof, an aptamer, at least one heavy or light chain CDR of a reference antibody molecule, and at least six CDRs from one or more reference antibody molecules.

Suitably, the one or more binding molecules or reporter molecules are antibodies or binding fragments thereof.

Suitably, the one or more binding molecules is an anti-IL-33 antibody or binding fragment thereof. Suitably, at least one of the binding molecules is an anti-oxidised IL-33 antibody or binding fragment thereof. Suitably, at least one of the binding molecules is an anti-reduced IL-33 antibody or binding fragment thereof. Suitably, at least one of the binding molecules is an anti-IL-33/sST2 antibody or binding fragment thereof.

Suitably, at least one of the reporter molecules is an anti-reduced-IL-33-binding molecule complex antibody or binding fragment thereof. Suitably, at least one of the reporter molecules is an anti-oxidised IL-33-binding molecule complex antibody or binding fragment thereof. Suitably, at least one of the reporter molecules is an anti-IL-33/sST2-binding molecule complex antibody or binding fragment thereof.

“Antibody” as employed herein refers to an immunoglobulin molecule as discussed below in more detail, in particular a full-length antibody or a molecule comprising a full-length antibody, for example a DVD-Ig mole and the like.

A “binding fragment thereof” is interchangeable with “antigen binding fragment thereof” and refers to an epitope/antigen binding fragment of an antibody fragment, for example comprising a binding region, in particular comprising 6 CDRs, such as 3 CDRs in heavy variable region and 3 CDRs in light variable region.

Suitably, the one or more binding molecules or reporter molecules are antibodies or antigen binding fragments thereof.

Encompassed by the term ‘antibody or antigen binding fragment thereof’ are any antibodies, or antigen binding fragments thereof that compete with an antibody of the invention for specific binding to an antigen. Suitably therefore the binding molecules or reporter molecules referred to herein, also encompass molecules which compete with said binding molecules or reporter molecules to bind to the relevant antigen. Suitably therefore the binding molecules or reporter molecules referred to herein, also encompass molecules which compete with said binding molecules or reporter molecules to specifically bind to reduced IL-33, oxidised IL-33, or IL-33/sST2. Such competitive antibodies or antigen binding fragments may be identified in a competitive binding assay well known in the art. Suitably competitive antibodies or antigen binding fragments substantially inhibit adhesion of a receptor to a counterreceptor, such as reduced IL-33 binding to an ST2 receptor. Suitably an excess of competitive antibody or antigen binding fragment reduces the quantity of receptor bound to counterreceptor by at least about 20%, 40%, 60% or 80%, and more usually greater than about 85% (as measured in an in vitro competitive binding assay).

The terms “complementarity determining regions” and “CDRs” as used herein refer to the amino acid residues of an antibody or antigen-binding fragment that are responsible for antigen binding.

Suitably, the antibody or binding fragment thereof is selected from: naturally-occurring, polyclonal, monoclonal, multispecific, mouse, human, humanized, primatized, or chimeric. Suitably, the antibody or binding fragment thereof may be an epitope-binding fragment, e.g., Fab′ and F(ab′)2, Fd, Fvs, single-chain Fvs (scFv), disulfide-linked Fvs (sdFv), fragments comprising either a VL or VH domain, or fragments produced by a Fab expression library. Suitably, the antibody or binding fragment thereof may be a minibody, a diabody, a triabody, a tetrabody, or a single chain antibody. Suitably, the antibody or binding fragment thereof is a monoclonal antibody. ScFv molecules are known in the art and are described, e.g., in U.S. Pat. No. 5,892,019. In one instance the one or more binding molecules or reporter molecules are Fab′ or F(ab′) 2 epitope-binding fragments.

The phrases “monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope Immunoglobulin or antibody molecules of the disclosure can be of any type (e.g., IgG, IgE, IgM, IgD, IgA, and IgY), class (e.g., IgG1, IgG2, IgG3, IgG4, IgAQ1, and IgA2, etc.), or subclass of immunoglobulin molecule.

Suitably the or each antibody or binding fragment thereof used as a binding molecule is a capture antibody.

Suitably the or each antibody or binding fragment thereof used as a reporter molecule is a probe antibody.

Suitably, as above, the binding molecules and reporter molecules are paired, therefore each capture antibody has a corresponding probe antibody. Suitably the capture antibodies are paired with probe antibodies.

Suitably, therefore, the one or more binding molecules is an anti-IL-33 capture antibody. Suitably, at least one of the binding molecules is an anti-oxidised IL-33 capture antibody. Suitably, at least one of the binding molecules is an anti-reduced IL-33 capture antibody. Suitably, at least one of the binding molecules is an anti-IL-33/sST2 capture antibody.

Suitably, at least one of the reporter molecules is an anti-reduced-IL-33 probe antibody. Suitably, at least one of the reporter molecules is an anti-oxidised IL-33 probe antibody. Suitably, at least one of the reporter molecules is an anti-IL-33/sST2 probe antibody or binding fragment thereof.

Suitably the contacting step of the methods comprises the use of one or more capture antibodies capable of binding to one or more antigenically distinct forms of IL-33 to form one or more complexes. Suitably the detecting step comprises the use of one or more probe antibodies capable of binding to the one or more complexes.

Suitably the one or more binding molecules comprises specific sequences which allow the binding molecule to bind to one of oxidised IL-33, reduced IL-33, or IL-33/sST2. Suitably the one or more reporter molecules comprises specific sequences which allow the reporter molecule to bind to one of oxidised IL-33, reduced IL-33, or IL-33/sST2 binding molecule complexes.

Suitable binding and reporter molecules having such capabilities, such as antibodies, are available in the art and may be ordered from reagent companies such as: R&D systems, for example.

However, suitably the binding molecules and/or reporter molecules used in the methods and kit of the present disclosure comprise the following sequences.

### Reduced IL-33 Binding Molecule-AB1070012

Suitably the binding molecule which binds to reduced IL-33 comprises a heavy chain variable region or domain (VH) and a light chain variable region or domain (VL). Suitably the binding molecule which binds to reduced IL-33 comprises 3 CDRs in a heavy chain variable region or domain (VH) and 3 CDRs in a light chain variable region or domain (VL). Suitably the binding molecule which binds to reduced IL-33 may comprise 3 CDRs, for example in a heavy chain variable region according to SEQ ID NO: 4 and/or may comprise 3 CDRs in a light chain variable region according to SEQ ID NO: 8.

Suitably the binding molecule which binds to reduced IL-33 comprises 3 CDRs in a heavy chain variable region (VH) wherein the VH has an amino acid sequence at least 85%, at least 90%, for example 91, 92, 93, 94, 95, 96, 97, 98, 99 or 100% identical to a VH according to SEQ ID NO: 4, and/or comprises 3 CDRs in a light chain variable region (VL) wherein the VL has an amino acid sequence at least 85%, at least 90%, for example 91, 92, 93, 94, 95, 96, 97, 98, 99 or 100% identical to a VL according to SEQ ID NO: 8.

Suitably the binding molecule which binds to reduced IL-33 may comprise a variable heavy domain (VH) and a variable light domain (VL), wherein the VH comprises VHCDRs 1-3 of: SEQ ID NO: 1, 2, and 3 wherein one or more VHCDRs have 3 or fewer single amino acid substitutions, deletions or insertions. Suitably such substitutions may be conservative substitutions.

Suitably, one or more VHCDRs have 3, 2, 1, or no single amino acid substitutions, deletions or insertions. Suitably the VHCDRs consist of SEQ ID NO: 1, 2, and 3.

Suitably the binding molecule which binds to reduced IL-33 may comprise a variable heavy domain (VH) and a variable light domain (VL), wherein the VL comprises VLCDRs 1-3 of: SEQ ID NO:5, 6 and 7 wherein one or more VLCDRs have 3 or fewer single amino acid substitutions, deletions or insertions. Suitably such substitutions may be conservative substitutions.

Suitably, one or more VLCDRs have 3, 2, 1, or no single amino acid substitutions, deletions or insertions. Suitably the VLCDRs consist of SEQ ID NO:5, 6 and 7.

Suitably the binding molecule which binds to reduced IL-33 may comprise a VH having VHCDRs 1-3 of: SEQ ID NO: 1, 2 and 3, and a VL having VLCDRs of SEQ ID NO: 5, 6 and 7, wherein one or more of the CDRs have 3 or fewer single amino acid substitutions, deletions or insertions. Suitably, one or more of the CDRs have 3, 2, 1, or no single amino acid substitutions, deletions or insertions. Suitably such substitutions may be conservative substitutions.

Suitably the binding molecule which binds to reduced IL-33 may comprise a VH having VHCDRs 1-3 of: SEQ ID NO: 1, 2 and 3, and a VL having VLCDRs of SEQ ID NO: 5, 6 and 7. Suitably the binding molecule which binds to reduced IL-33 may comprise a VH having VHCDRs 1-3 consisting of: SEQ ID NO: 1, 2 and 3, and a VL having VLCDRs consisting of SEQ ID NO: 5, 6 and 7.

Suitably the binding molecule which binds to reduced IL-33 may comprise CDRs selected from one or more of: a VHCDR1 having the sequence of: SEQ ID NO:1, a VHCDR2 having the sequence of: SEQ ID NO:2, a VHCDR3 having the sequence of: SEQ ID NO: 3, a VLCDR1 having the sequence of: SEQ ID NO:5, a VLCDR2 having the sequence of: SEQ ID NO:6, and a VLCDR3 having the sequence of: SEQ ID NO:7.

Suitably the binding molecule which binds to reduced IL-33 may comprise the following CDRs: a VHCDR1 having the sequence of: SEQ ID NO:1, a VHCDR2 having the sequence of: SEQ ID NO: 2, a VHCDR3 having the sequence of: SEQ ID NO: 3, a VLCDR1 having the sequence of: SEQ ID NO:5, a VLCDR2 having the sequence of: SEQ ID NO:6, and a VLCDR3 having the sequence of: SEQ ID NO:7.

Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 4.

Suitably, therefore the binding molecule comprises a VH, wherein a VH disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 4, and comprises VHCDRs 1-3 of: SEQ ID NO: 1, 2, and 3, wherein one or more VHCDRs have 3 or fewer single amino acid substitutions, deletions or insertions. Suitably, one or more VHCDRs have 3, 2, 1, or no single amino acid substitutions, deletions or insertions.

Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 4, and comprises VHCDRs 1-3 of: SEQ ID NO: 1, 2, and 3.

Suitably therefore, the binding molecule which binds to reduced IL-33 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 8.

Suitably, therefore the binding molecule comprises a VL, wherein a VL disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the binding molecule which binds to reduced IL-33 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 8, and comprises VLCDRs 1-3 of: SEQ ID NO:5, 6 and 7 wherein one or more VLCDRs have 3 or fewer single amino acid substitutions, deletions or insertions.

Suitably, one or more VLCDRs have 3, 2, 1, or no single amino acid substitutions, deletions or insertions.

Suitably therefore, the binding molecule which binds to reduced IL-33 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 8, and comprises VLCDRs 1-3 of: SEQ ID NO:5, 6 and 7.

Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 4, and comprises VHCDRs 1-3 of: SEQ ID NO: 1, 2, and 3, and wherein the VL has an amino acid sequence at least 85%, at least 90%, for example 91, 92, 93, 94, 95, 96, 97, 98, 99 or 100% identical to a VL according to SEQ ID NO: 8, and comprises VLCDRs 1-3 of: SEQ ID NO:5, 6 and 7.

Suitably therefore, the binding molecule which binds to reduced IL-33 is an antibody or binding fragment thereof comprising a VH, wherein the VH comprises an amino acid sequence according to SEQ ID NO:4. Suitably therefore, the binding molecule which binds to reduced IL-33 is an antibody or binding fragment thereof comprising a VL, wherein the VL comprises an amino acid sequence according to SEQ ID NO:8. Suitably therefore, the binding molecule which binds to reduced IL-33 is an antibody or binding fragment thereof comprising a VH and a VL, wherein the VH has an amino acid sequence consisting of SEQ ID NO: 4, and the VL has an amino acid sequence consisting of SEQ ID NO: 8.

### Reduced IL-33 Binding Molecule 2-AB1070019

Suitably the binding molecule which binds to reduced IL-33 comprises a heavy chain variable region or domain (VH) and a light chain variable region or domain (VL). Suitably the binding molecule which binds to reduced IL-33 comprises 3 CDRs in a heavy chain variable region or domain (VH) and 3 CDRs in a light chain variable region or domain (VL). Suitably the binding molecule which binds to reduced IL-33 may comprise 3 CDRs, for example in a heavy chain variable region according to SEQ ID NO: 28 and/or may comprise 3 CDRs in a light chain variable region according to SEQ ID NO: 32.

Suitably the binding molecule which binds to reduced IL-33 comprises 3 CDRs in a heavy chain variable region (VH) wherein the VH has an amino acid sequence at least 85%, at least 90%, for example 91, 92, 93, 94, 95, 96, 97, 98, 99 or 100% identical to a VH according to SEQ ID NO: 28, and/or comprises 3 CDRs in a light chain variable region (VL) wherein the VL has an amino acid sequence at least 85%, at least 90%, for example 91, 92, 93, 94, 95, 96, 97, 98, 99 or 100% identical to a VL according to SEQ ID NO: 32.

Suitably the binding molecule which binds to reduced IL-33 may comprise a variable heavy domain (VH) and a variable light domain (VL) wherein the VH comprises VHCDRs 1-3 of: SEQ ID NO: 25, 26 and 27 wherein one or more VHCDRs have 3 or fewer single amino acid substitutions, deletions or insertions.

Suitably, one or more VHCDRs may comprise 3, 2, 1, or no single amino acid substitutions, deletions or insertions. Suitably the VHCDRs consist of SEQ ID NO: 25, 26 and 27.

Suitably the binding molecule which binds to reduced IL-33 may comprise a variable heavy domain (VH) and a variable light domain (VL) wherein the VL comprises VLCDRs 1-3 of: SEQ ID NO: 29, 30 and 31 wherein one or more VLCDRs have 3 or fewer single amino acid substitutions, deletions or insertions.

Suitably, one or more VLCDRs may comprise 3, 2, 1, or no single amino acid substitutions, deletions or insertions. Suitably the VLCDRs consist of SEQ ID NO: 29, 30, and 31.

Suitably the binding molecule which binds to reduced IL-33 may comprise a VH having VHCDRs 1-3 of: SEQ ID NO: 25, 26 and 27, and a VL having VLCDRs of SEQ ID NO: 29, 30 and 31, wherein one or more of the CDRs have 3 or fewer single amino acid substitutions, deletions or insertions. Suitably, one or more of the CDRs have 3, 2, 1, or no single amino acid substitutions, deletions or insertions. Suitably such substitutions may be conservative substitutions.

Suitably the binding molecule which binds to reduced IL-33 may comprise a VH having VHCDRs 1-3 of: SEQ ID NO: 25, 26 and 27 and a VL having VLCDRs of SEQ ID NO: 29, 30 and 31. Suitably the binding molecule which binds to reduced IL-33 may comprise a VH having VHCDRs 1-3 consisting of: SEQ ID NO: 25, 26 and 27, and a VL having VLCDRs consisting of SEQ ID NO: 29, 30 and 31.

Suitably the binding molecule which binds to reduced IL-33 may comprise CDRs selected from: a VHCDR1 having the sequence of: SEQ ID NO:25 a VHCDR2 having the sequence of: SEQ ID NO:26 a VHCDR3 having the sequence of: SEQ ID NO:27 a VLCDR1 having the sequence of: SEQ ID NO:29 a VLCDR2 having the sequence of: SEQ ID NO:30 and a VLCDR3 having the sequence of: SEQ ID NO:31.

Suitably the binding molecule which binds to reduced IL-33 may comprise the following CDRs: a VHCDR1 having the sequence of: SEQ ID NO:25, a VHCDR2 having the sequence of: SEQ ID NO: 26, a VHCDR3 having the sequence of: SEQ ID NO: 27, a VLCDR1 having the sequence of: SEQ ID NO:29, a VLCDR2 having the sequence of: SEQ ID NO:30, and a VLCDR3 having the sequence of: SEQ ID NO:31.Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 28.

Suitably, therefore the binding molecule comprises a VH, wherein a VH disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 28, and comprises VHCDRs 1-3 of: SEQ ID NO: 25, 26 and 27, wherein one or more VHCDRs have 3 or fewer single amino acid substitutions, deletions or insertions. Suitably, one or more VHCDRs have 3, 2, 1, or no single amino acid substitutions, deletions or insertions.

Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 28, and comprises VHCDRs 1-3 of: SEQ ID NO: 25, 26 and 27.

Suitably therefore, the binding molecule which binds to reduced IL-33 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 32.

Suitably, therefore the binding molecule comprises a VL, wherein a VL disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the binding molecule which binds to reduced IL-33 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 32, and comprises VLCDRs 1-3 of: SEQ ID NO: 29, 30 and 31 wherein one or more VLCDRs have 3 or fewer single amino acid substitutions, deletions or insertions. Suitably, one or more VLCDRs have 3, 2, 1, or no single amino acid substitutions, deletions or insertions.

Suitably therefore, the binding molecule which binds to reduced IL-33 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 32, and comprises VLCDRs 1-3 of: SEQ ID NO:29, 30 and 31.

Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 28, and comprises VHCDRs 1-3 of: SEQ ID NO: 25, 26 and 27, and wherein the VL has an amino acid sequence at least 85%, at least 90%, for example 91, 92, 93, 94, 95, 96, 97, 98, 99 or 100% identical to a VL according to SEQ ID NO: 32, and comprises VLCDRs 1-3 of: SEQ ID NO:29, 30 and 31.

Suitably therefore, the binding molecule which binds to reduced IL-33 is an antibody or binding fragment thereof comprising a VH, wherein the VH comprises an amino acid sequence according to SEQ ID NO:28. Suitably therefore, the binding molecule which binds to reduced IL-33 is an antibody or binding fragment thereof comprising a VL, wherein the VL comprises an amino acid sequence according to SEQ ID NO:32.

Suitably therefore, the binding molecule which binds to reduced IL-33 is an antibody or binding fragment thereof comprising a VH and a VL, wherein the VH has an amino acid sequence consisting of SEQ ID NO: 28, and the VL has an amino acid sequence consisting of SEQ ID NO: 32.

### Reduced IL-33 Binding Molecule 3-AB1070069

Suitably the binding molecule which binds to reduced IL-33 comprises a heavy chain variable region or domain (VH) and a light chain variable region or domain (VL). Suitably the binding molecule which binds to reduced IL-33 comprises 3 CDRs in a heavy chain variable region or domain (VH) and 3 CDRs in a light chain variable region or domain (VL). Suitably the binding molecule which binds to reduced IL-33 may comprise 3 CDRs, for example in a heavy chain variable region according to SEQ ID NO: 38 and/or may comprise 3 CDRs in a light chain variable region according to SEQ ID NO: 42.

Suitably the binding molecule which binds to reduced IL-33 comprises 3 CDRs in a heavy chain variable region (VH) wherein the VH has an amino acid sequence at least 85%, at least 90%, for example 91, 92, 93, 94, 95, 96, 97, 98, 99 or 100% identical to a VH according to SEQ ID NO: 38, and/or comprises 3 CDRs in a light chain variable region (VL) wherein the VL has an amino acid sequence at least 85%, at least 90%, for example 91, 92, 93, 94, 95, 96, 97, 98, 99 or 100% identical to a VL according to SEQ ID NO: 42.

Suitably the binding molecule which binds to reduced IL-33 may comprise a variable heavy domain (VH) and a variable light domain (VL), wherein the VH comprises VHCDRs 1-3 of: SEQ ID NO: 35, 36 and 37 wherein one or more VHCDRs have 3 or fewer single amino acid substitutions, deletions or insertions.

Suitably, one or more VHCDRs may comprise 3, 2, 1, or no single amino acid substitutions, deletions or insertions. Suitably the VHCDRs consist of SEQ ID NO: 35, 36 and 37.

Suitably the binding molecule which binds to reduced IL-33 may comprise a variable heavy domain (VH) and a variable light domain (VL), wherein the VL comprises VLCDRs 1-3 of: SEQ ID NO: 39, 40 and 41 wherein one or more VLCDRs have 3 or fewer single amino acid substitutions, deletions or insertions.

Suitably, one or more VLCDRs may comprise 3, 2, 1, or no single amino acid substitutions, deletions or insertions. Suitably the VLCDRs consist of SEQ ID NO: 39, 40, and 41.

Suitably the binding molecule which binds to reduced IL-33 may comprise a VH having VHCDRs 1-3 of: SEQ ID NO: 35, 36 and 37, and a VL having VLCDRs of SEQ ID NO: 39, 40 and 41, wherein one or more of the CDRs have 3 or fewer single amino acid substitutions, deletions or insertions. Suitably, one or more of the CDRs have 3, 2, 1, or no single amino acid substitutions, deletions or insertions. Suitably such substitutions may be conservative substitutions.

Suitably the binding molecule which binds to reduced IL-33 may comprise a VH having VHCDRs 1-3 of: SEQ ID NO: 35, 36 and 37 and a VL having VLCDRs of SEQ ID NO: 39, 40 and 41. Suitably the binding molecule which binds to reduced IL-33 may comprise a VH having VHCDRs 1-3 consisting of: SEQ ID NO: 35, 36 and 37, and a VL having VLCDRs consisting of SEQ ID NO: 39, 40 and 41.

Suitably the binding molecule which binds to reduced IL-33 may comprise CDRs selected from: a VHCDR1 having the sequence of: SEQ ID NO:35 a VHCDR2 having the sequence of: SEQ ID NO:36 a VHCDR3 having the sequence of: SEQ ID NO:37 a VLCDR1 having the sequence of: SEQ ID NO:39 a VLCDR2 having the sequence of: SEQ ID NO:40 and a VLCDR3 having the sequence of: SEQ ID NO:41.

Suitably the binding molecule which binds to reduced IL-33 may comprise the following CDRs: a VHCDR1 having the sequence of: SEQ ID NO:35, a VHCDR2 having the sequence of: SEQ ID NO: 36, a VHCDR3 having the sequence of: SEQ ID NO: 37, a VLCDR1 having the sequence of: SEQ ID NO:39, a VLCDR2 having the sequence of: SEQ ID NO:40, and a VLCDR3 having the sequence of: SEQ ID NO:41.

Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 38.

Suitably, therefore the binding molecule comprises a VH, wherein a VH disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 38, and comprises VHCDRs 1-3 of: SEQ ID NO: 35, 36 and 37, wherein one or more VHCDRs have 3 or fewer single amino acid substitutions, deletions or insertions. Suitably, one or more VHCDRs have 3, 2, 1, or no single amino acid substitutions, deletions or insertions.

Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 38, and comprises VHCDRs 1-3 of: SEQ ID NO: 35, 36 and 37.

Suitably therefore, the binding molecule which binds to reduced IL-33 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 42.

Suitably, therefore the binding molecule comprises a VL, wherein a VL disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the binding molecule which binds to reduced IL-33 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 42, and comprises VLCDRs 1-3 of: SEQ ID NO: 39, 40 and 41 wherein one or more VLCDRs have 3 or fewer single amino acid substitutions, deletions or insertions. Suitably, one or more VLCDRs have 3, 2, 1, or no single amino acid substitutions, deletions or insertions.

Suitably therefore, the binding molecule which binds to reduced IL-33 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 42, and comprises VLCDRs 1-3 of: SEQ ID NO:39, 40 and 41.

Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 38, and comprises VHCDRs 1-3 of: SEQ ID NO: 35, 36 and 37, and wherein the VL has an amino acid sequence at least 85%, at least 90%, for example 91, 92, 93, 94, 95, 96, 97, 98, 99 or 100% identical to a VL according to SEQ ID NO: 42, and comprises VLCDRs 1-3 of: SEQ ID NO:39, 40 and 41.

Suitably therefore, the binding molecule which binds to reduced IL-33 is an antibody or binding fragment thereof comprising a VH, wherein the VH comprises an amino acid sequence according to SEQ ID NO: 38. Suitably therefore, the binding molecule which binds to reduced IL-33 is an antibody or binding fragment thereof comprising a VL, wherein the VL comprises an amino acid sequence according to SEQ ID NO:42.

Suitably therefore, the binding molecule which binds to reduced IL-33 is an antibody or binding fragment thereof comprising a VH and a VL, wherein the VH has an amino acid sequence consisting of SEQ ID NO: 38, and the VL has an amino acid sequence consisting of SEQ ID NO: 42.

### Oxidised IL-33 Binding Molecule-AB1070141

Suitably the binding molecule which binds to oxidised IL-33 comprises a heavy chain variable region or domain (VH) and a light chain variable region or domain (VL). Suitably the binding molecule which binds to oxidised IL-33 comprises 3 CDRs in a heavy chain variable region or domain (VH) and 3 CDRs in a light chain variable region or domain (VL). Suitably the binding molecule which binds to oxidised IL-33 may comprise 3 CDRs, for example in a heavy chain variable region according to SEQ ID NO: 12 and/or may comprise 3 CDRs in a light chain variable region according to SEQ ID NO: 16.

Suitably the binding molecule which binds to oxidised IL-33 comprises 3 CDRs in a heavy chain variable region (VH) wherein the VH has an amino acid sequence at least 85%, at least 90%, for example 91, 92, 93, 94, 95, 96, 97, 98, 99 or 100% identical to a VH according to SEQ ID NO: 12, and/or comprises 3 CDRs in a light chain variable region (VL) wherein the VL has an amino acid sequence at least 85%, at least 90%, for example 91, 92, 93, 94, 95, 96, 97, 98, 99 or 100% identical to a VL according to SEQ ID NO: 16.

Suitably the binding molecule which binds to oxidised IL-33 may comprise a variable heavy domain (VH) and a variable light domain (VL) wherein the VH comprises VHCDRs 1-3 of: SEQ ID NO: 9, 10 and 11 wherein one or more VHCDRs have 3 or fewer single amino acid substitutions, deletions or insertions.

Suitably, one or more VHCDRs may comprise 3, 2, 1, or no single amino acid substitutions, deletions or insertions. Suitably the VHCDRs consist of SEQ ID NO: 9, 10 and 11.

Suitably the binding molecule which binds to oxidised IL-33 may comprise a variable heavy domain (VH) and a variable light domain (VL) wherein the VL comprises VLCDRs 1-3 of: SEQ ID NO: 13, 14 and 15 wherein one or more VLCDRs have 3 or fewer single amino acid substitutions, deletions or insertions.

Suitably, one or more VLCDRs may comprise 3, 2, 1, or no single amino acid substitutions, deletions or insertions. Suitably the VLCDRs consist of SEQ ID NO: 13, 14 and 15.

Suitably the binding molecule which binds to oxidised IL-33 may comprise a VH having VHCDRs 1-3 of: SEQ ID NO: 9, 10 and 11, and a VL having VLCDRs of SEQ ID NO: 13, 14 and 15, wherein one or more of the CDRs have 3 or fewer single amino acid substitutions, deletions or insertions. Suitably, one or more of the CDRs have 3, 2, 1, or no single amino acid substitutions, deletions or insertions. Suitably such substitutions may be conservative substitutions.

Suitably the binding molecule which binds to oxidised IL-33 may comprise a VH having VHCDRs 1-3 of: SEQ ID NO: 9, 10 and 11 and a VL having VLCDRs of SEQ ID NO: 13, 14 and 15. Suitably the binding molecule which binds to oxidised IL-33 may comprise a VH having VHCDRs 1-3 consisting of: SEQ ID NO: 9, 10 and 11, and a VL having VLCDRs consisting of SEQ ID NO: 13, 14 and 15.

Suitably the binding molecule which binds to oxidised IL-33 may comprise one or more CDRs selected from: a VHCDR1 having the sequence of: SEQ ID NO:9 a VHCDR2 having the sequence of: SEQ ID NO:10 a VHCDR3 having the sequence of: SEQ ID NO:11 a VLCDR1 having the sequence of: SEQ ID NO:13 a VLCDR2 having the sequence of: SEQ ID NO: 14 and a VLCDR3 having the sequence of: SEQ ID NO:15.

Suitably the binding molecule which binds to reduced IL-33 may comprise the following CDRs: a VHCDR1 having the sequence of: SEQ ID NO:9, a VHCDR2 having the sequence of: SEQ ID NO: 10, a VHCDR3 having the sequence of: SEQ ID NO: 11, a VLCDR1 having the sequence of: SEQ ID NO:13, a VLCDR2 having the sequence of: SEQ ID NO: 14, and a VLCDR3 having the sequence of: SEQ ID NO: 15.

Suitably therefore, the binding molecule which binds to oxidised IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 12.

Suitably, therefore the binding molecule comprises a VH, wherein a VH disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the binding molecule which binds to oxidised IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 12, and comprises VHCDRs 1-3 of: SEQ ID NO: 9, 10 and 11, wherein one or more VHCDRs have 3 or fewer single amino acid substitutions, deletions or insertions. Suitably, one or more VHCDRs have 3, 2, 1, or no single amino acid substitutions, deletions or insertions.

Suitably therefore, the binding molecule which binds to reduced IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 12, and comprises VHCDRs 1-3 of: SEQ ID NO: 9, 10 and 11.

Suitably therefore, the binding molecule which binds to oxidised IL-33 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 16.

Suitably, therefore the binding molecule comprises a VL, wherein a VL disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the binding molecule which binds to oxidised IL-33 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 16, and comprises VLCDRs 1-3 of: SEQ ID NO: 13, 14 and 15 wherein one or more VLCDRs have 3 or fewer single amino acid substitutions, deletions or insertions. Suitably, one or more VLCDRs have 3, 2, 1, or no single amino acid substitutions, deletions or insertions.

Suitably therefore, the binding molecule which binds to oxidised IL-33 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 16, and comprises VLCDRs 1-3 of: SEQ ID NO: 13, 14 and 15.

Suitably therefore, the binding molecule which binds to oxidised IL-33 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 12, and comprises VHCDRs 1-3 of: SEQ ID NO: 9, 10 and 11, and wherein the VL has an amino acid sequence at least 85%, at least 90%, for example 91, 92, 93, 94, 95, 96, 97, 98, 99 or 100% identical to a VL according to SEQ ID NO: 16, and comprises VLCDRs 1-3 of: SEQ ID NO: 13, 14 and 15.

Suitably therefore, the binding molecule which binds to oxidised IL-33 is an antibody or binding fragment thereof comprising a VH, wherein the VH comprises an amino acid sequence according to SEQ ID NO:12. Suitably therefore, the binding molecule which binds to oxidised IL-33 is an antibody or binding fragment thereof comprising a VL, wherein the VL comprises an amino acid sequence according to SEQ ID NO:16.

Suitably therefore, the binding molecule which binds to oxidised IL-33 is an antibody or binding fragment thereof comprising a VH and a VL, wherein the VH has an amino acid sequence consisting of SEQ ID NO: 12, and the VL has an amino acid sequence consisting of SEQ ID NO: 16.

### IL33/sST2 Binding Molecule-AB1070008

Suitably the binding molecule which binds to IL-33/sST2 comprises a heavy chain variable region or domain (VH) and a light chain variable region or domain (VL). Suitably the binding molecule which binds to IL-33/sST2 comprises 3 CDRs in a heavy chain variable region or domain (VH) and 3 CDRs in a light chain variable region or domain (VL). Suitably the binding molecule which binds to IL-33/sST2 may comprise 3 CDRs, for example in a heavy chain variable region according to SEQ ID NO:20 and/or may comprise 3 CDRs in a light chain variable region according to SEQ ID NO:24.

Suitably the binding molecule which binds to IL-33/sST2 comprises 3 CDRs in a heavy chain variable region (VH) wherein the VH has an amino acid sequence at least 85%, at least 90%, for example 91, 92, 93, 94, 95, 96, 97, 98, 99 or 100% identical to a VH according to SEQ ID NO: 20, and/or comprises 3 CDRs in a light chain variable region wherein the VL has an amino acid sequence at least 90%, for example 91, 92, 93, 94, 95, 96, 97, 98, 99 or 100% identical to a VL according to SEQ ID NO: 24.

Suitably the binding molecule which binds to IL-33/sST2 may comprise a variable heavy domain (VH) and a variable light domain (VL) wherein the VH comprises VHCDRs 1-3 of: SEQ ID NO: 17, 18 and 19 wherein one or more VHCDRs have 3 or fewer single amino acid substitutions, deletions or insertions.

Suitably, one or more VHCDRs comprise 3, 2, 1, or no single amino acid substitutions, deletions or insertions. Suitably the VHCDRs consist of SEQ ID NO: 17, 18 and 19.

Suitably the binding molecule which binds to IL-33/sST2 may comprise a variable heavy domain (VH) and a variable light domain (VL) wherein the VL comprises VLCDRs 1-3 of: SEQ ID NO: 21, 22 and 23 wherein one or more VLCDRs have 3 or fewer single amino acid substitutions, deletions or insertions.

Suitably, one or more VLCDRs comprise 3, 2, 1, or no single amino acid substitutions, deletions or insertions. Suitably the VLCDRs consist of SEQ ID NO: 21, 22 and 23.

Suitably the binding molecule which binds to IL-33/sST2 may comprise a VH having VHCDRs 1-3 of: SEQ ID NO: 17, 18 and 19, and a VL having VLCDRs of SEQ ID NO: 21, 22 and 23, wherein one or more of the CDRs have 3 or fewer single amino acid substitutions, deletions or insertions. Suitably, one or more of the CDRs have 3, 2, 1, or no single amino acid substitutions, deletions or insertions. Suitably such substitutions may be conservative substitutions.

Suitably the binding molecule which binds to IL-33/sST2 may comprise a VH having VHCDRs 1-3 of: SEQ ID NO: 17, 18 and 19 and a VL having VLCDRs of SEQ ID NO: 21, 22 and 23. Suitably the binding molecule which binds to IL-33/sST2 may comprise a VH having VHCDRs 1-3 consisting of: SEQ ID NO: 17, 18 and 19, and a VL having VLCDRs consisting of SEQ ID NO: 21, 22 and 23.

Suitably the binding molecule which binds to IL-33/sST2 may comprise any of the following CDRs: a VHCDR1 having the sequence of: SEQ ID NO:17, a VHCDR2 having the sequence of: SEQ ID NO:18, a VHCDR3 having the sequence of: SEQ ID NO: 19, a VLCDR1 having the sequence of: SEQ ID NO:21, a VLCDR2 having the sequence of: SEQ ID NO:22, and a VLCDR3 having the sequence of: SEQ ID NO:23.

Suitably the binding molecule which binds to IL-33/sST2 may comprise a VHCDR1 having the sequence of: SEQ ID NO: 17 a VHCDR2 having the sequence of: SEQ ID NO: 18 a VHCDR3 having the sequence of: SEQ ID NO:19 a VLCDR1 having the sequence of: SEQ ID NO:21 a VLCDR2 having the sequence of: SEQ ID NO:22 and a VLCDR3 having the sequence of: SEQ ID NO:23.

Suitably therefore, the binding molecule which binds to IL-33/sST2 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 20.

Suitably, therefore the binding molecule comprises a VH, wherein a VH disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the binding molecule which binds to IL-33/sST2 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 20, and comprises VHCDRs 1-3 of: SEQ ID NO: 17, 18 and 19, wherein one or more VHCDRs have 3 or fewer single amino acid substitutions, deletions or insertions. Suitably, one or more VHCDRs have 3, 2, 1, or no single amino acid substitutions, deletions or insertions.

Suitably therefore, the binding molecule which binds to IL-33/sST2 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 20, and comprises VHCDRs 1-3 of: SEQ ID NO: 17, 18 and 19.

Suitably therefore, the binding molecule which binds to IL-33/sST2 is an antibody or binding fragment thereof comprising a VH and VL, wherein the VL has an amino acid sequence at least 90%, for example 91, 92, 93, 94, 95, 96, 97, 98, 99 or 100% identical to a VL according to SEQ ID NO: 24.

Suitably, therefore the binding molecule comprises a VL, wherein a VL disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the binding molecule which binds to IL-33/sST2 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 24, and comprises VLCDRs 1-3 of: SEQ ID NO: 21, 22 and 23 wherein one or more VLCDRs have 3 or fewer single amino acid substitutions, deletions or insertions. Suitably, one or more VLCDRs have 3, 2, 1, or no single amino acid substitutions, deletions or insertions.

Suitably therefore, the binding molecule which binds to IL-33/sST2 is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 24, and comprises VLCDRs 1-3 of: SEQ ID NO:21, 22 and 23.

Suitably therefore, the binding molecule which binds to IL-33/sST2 is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 20, and comprises VHCDRs 1-3 of: SEQ ID NO: 17, 18 and 19, and wherein the VL has an amino acid sequence at least 85%, at least 90%, for example 91, 92, 93, 94, 95, 96, 97, 98, 99 or 100% identical to a VL according to SEQ ID NO: 24, and comprises VLCDRs 1-3 of: SEQ ID NO:21, 22 and 23.

Suitably therefore, the binding molecule which binds to oxidised IL-33 is an antibody or binding fragment thereof comprising a VH, wherein the VH comprises an amino acid sequence according to SEQ ID NO:20. Suitably therefore, the binding molecule which binds to oxidised IL-33 is an antibody or binding fragment thereof comprising a VL, wherein the VL comprises an amino acid sequence according to SEQ ID NO:24.

Suitably therefore, the binding molecule which binds to IL-33/sST2 is an antibody or binding fragment thereof comprising a VH and a VL, wherein the VH has an amino acid sequence consisting of SEQ ID NO:20, and the VL has an amino acid sequence consisting of SEQ ID NO: 24.

### Reduced IL-33 Reporter Molecule-AB1070019

Optionally, the reduced IL-33 binding molecule may be used as a reduced IL-33 reporter molecule. Suitably, the binding molecule of (b) (i) b of the first aspect or the binding molecule of (b) of the fourth aspect may also be used as a reporter molecule. Suitably therefore, in some assays for detecting reduced IL-33, the binding molecule and the reporter molecule may be the same. Suitably therefore, in some assays for detecting reduced IL-33, the binding molecule and the reporter molecule may both be the molecule as defined in step (b)ib of the first aspect or (b) of the fourth aspect.

Suitably the reporter molecule which binds to reduced-IL33-binding molecule complexes comprises a heavy chain variable region or domain (VH) and a light chain variable region or domain (VL). Suitably the reporter molecule which binds to reduced-IL33-binding molecule complexes comprises 3 CDRs in a heavy chain variable region or domain (VH) and 3 CDRs in a light chain variable region or domain (VL). Suitably reporter molecule which binds to reduced-IL33-binding molecule complexes may comprise 3 CDRs, for example in a heavy chain variable region according to SEQ ID NO: 28 and/or may comprise 3 CDRs in a light chain variable region according to SEQ ID NO:32.

Suitably the reporter molecule which binds to reduced-IL33-binding molecule complexes comprises 3 CDRs in a heavy chain variable region (VH) wherein the VH has an amino acid sequence at least 85%, at least 90%, for example 91, 92, 93, 94, 95, 96, 97, 98, 99 or 100% identical to a VH according to SEQ ID NO: 28, and/or comprises 3 CDRs in a light chain variable region (VL) wherein the VL has an amino acid sequence at least 85%, at least 90%, for example 91, 92, 93, 94, 95, 96, 97, 98, 99 or 100% identical to a VL according to SEQ ID NO: 32.

Suitably the reporter molecule which binds to reduced-IL33-binding molecule complexes may comprise a variable heavy domain (VH) and a variable light domain (VL) having VHCDRs 1-3 of: SEQ ID NO: 25, 26 and 27 wherein one or more VHCDRs have 3 or fewer single amino acid substitutions, deletions or insertions.

Suitably, one or more of the VHCDRs comprise 3, 2, 1, or no single amino acid substitutions, deletions or insertions. Suitably the VHCDRs consist of SEQ ID NO: 25, 26 and 27.

Suitably the reporter molecule which binds to reduced-IL33-binding molecule complexes may comprise a variable heavy domain (VH) and a variable light domain (VL) having VLCDRs 1-3 of: SEQ ID NO: 29, 30 and 31 wherein one or more VLCDRs have 3 or fewer single amino acid substitutions, deletions or insertions.

Suitably, one or more of the VLCDRs comprise 3, 2, 1, or no single amino acid substitutions, deletions or insertions. Suitably the VLCDRs consist of SEQ ID NO: 29, 30 and 31.

Suitably the reporter molecule which binds to reduced-IL33-binding molecule complexes may comprise a VH having VHCDRs 1-3 of: SEQ ID NO: 25, 26 and 27, and a VL having VLCDRs of SEQ ID NO: 29, 30 and 31, wherein one or more of the CDRs have 3 or fewer single amino acid substitutions, deletions or insertions. Suitably, one or more of the CDRs have 3, 2, 1, or no single amino acid substitutions, deletions or insertions. Suitably such substitutions may be conservative substitutions.

Suitably the reporter molecule which binds to reduced-IL33-binding molecule complexes may comprise a VH having VHCDRs 1-3 of: SEQ ID NO: 25, 26 and 27 and a VL having VLCDRs of SEQ ID NO: 29, 30 and 31. Suitably the reporter molecule which binds to reduced-IL33-binding molecule complexes may comprise a VH having VHCDRs 1-3 consisting of: SEQ ID NO: 25, 26 and 27, and a VL having VLCDRs consisting of SEQ ID NO: 29, 30 and 31.

Suitably the reporter molecule which binds to reduced-IL33-binding molecule complexes may comprise any of the following CDRs: a VHCDR1 having the sequence of: SEQ ID NO:25, a VHCDR2 having the sequence of: SEQ ID NO:26, a VHCDR3 having the sequence of: SEQ ID NO: 27, a VLCDR1 having the sequence of: SEQ ID NO:29, a VLCDR2 having the sequence of: SEQ ID NO:30, and a VLCDR3 having the sequence of: SEQ ID NO:31.

Suitably the reporter molecule which binds to reduced-IL33-binding molecule complexes may comprise a VHCDR1 having the sequence of: SEQ ID NO: 25 a VHCDR2 having the sequence of: SEQ ID NO: 26 a VHCDR3 having the sequence of: SEQ ID NO: 27 a VLCDR1 having the sequence of: SEQ ID NO: 29 a VLCDR2 having the sequence of: SEQ ID NO: 30 and a VLCDR3 having the sequence of: SEQ ID NO: 31.

Suitably therefore, the reporter molecule which binds to reduced-IL33-binding molecule complexes is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO:28.

Suitably, therefore the reporter molecule comprises a VH, wherein a VH disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the reporter molecule which binds to reduced-IL33-binding molecule complexes is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 28, and comprises VHCDRs 1-3 of: SEQ ID NO: 25, 26 and 27, wherein one or more VHCDRs have 3 or fewer single amino acid substitutions, deletions or insertions. Suitably, one or more VHCDRs have 3, 2, 1, or no single amino acid substitutions, deletions or insertions.

Suitably therefore, the reporter molecule which binds to reduced-IL33-binding molecule complexes is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 28, and comprises VHCDRs 1-3 of: SEQ ID NO: 25, 26 and 27.

Suitably therefore, the reporter molecule which binds to reduced-IL33-binding molecule complexes is an antibody or binding fragment thereof comprising a VH and VL, wherein the VL has an amino acid sequence at least 90%, for example 91, 92, 93, 94, 95, 96, 97, 98, 99 or 100% identical to a VL according to SEQ ID NO:32.

Suitably, therefore the reporter molecule comprises a VL, wherein a VL disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the reporter molecule which binds to reduced-IL33-binding molecule complexes is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 32, and comprises VLCDRs 1-3 of: SEQ ID NO: 29, 30 and 31 wherein one or more VLCDRs have 3 or fewer single amino acid substitutions, deletions or insertions. Suitably, one or more VLCDRs have 3, 2, 1, or no single amino acid substitutions, deletions or insertions.

Suitably therefore, the reporter molecule which binds to reduced-IL33-binding molecule complexes is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 32, and comprises VLCDRs 1-3 of: SEQ ID NO:29, 30 and 31.

Suitably therefore, the reporter molecule which binds to reduced-IL33-binding molecule complexes is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 28, and comprises VHCDRs 1-3 of: SEQ ID NO: 25, 26 and 27, and wherein the VL has an amino acid sequence at least 85%, at least 90%, for example 91, 92, 93, 94, 95, 96, 97, 98, 99 or 100% identical to a VL according to SEQ ID NO: 32, and comprises VLCDRs 1-3 of: SEQ ID NO: 29, 30 and 31.

Suitably therefore, the reporter molecule which binds to reduced-IL33-binding molecule complexes is an antibody or binding fragment thereof comprising a VH, wherein the VH comprises an amino acid sequence according to SEQ ID NO:28. Suitably therefore, the reporter molecule which binds to reduced-IL33-binding molecule complexes is an antibody or binding fragment thereof comprising a VL, wherein the VL comprises an amino acid sequence according to SEQ ID NO:32.

Suitably therefore, the reporter molecule which binds to reduced-IL33-binding molecule complexes is an antibody or binding fragment thereof comprising a VH and a VL, wherein the VH has an amino acid sequence consisting of SEQ ID NO: 28, and the VL has an amino acid sequence consisting of SEQ ID NO: 32.

### Reduced IL-33 Reporter Molecule 2-AB1070069

Optionally, the reduced IL-33 binding molecule may be used as a reduced IL-33 reporter molecule. Suitably, the binding molecule of (b)(i)b of the first aspect or the binding molecule of (b) of the fourth aspect may also be used as a reporter molecule. Suitably therefore, in some assays for detecting reduced IL-33, the binding molecule and the reporter molecule may be the same. Suitably therefore, in some assays for detecting reduced IL-33, the binding molecule and the reporter molecule may both be the molecule as defined in step (b)ib of the first aspect or (b) of the fourth aspect.

Suitably the reporter molecule which binds to reduced-IL33-binding molecule complexes comprises a heavy chain variable region or domain (VH) and a light chain variable region or domain (VL). Suitably the reporter molecule which binds to reduced-IL33-binding molecule complexes comprises 3 CDRs in a heavy chain variable region or domain (VH) and 3 CDRs in a light chain variable region or domain (VL). Suitably reporter molecule which binds to reduced-IL33-binding molecule complexes may comprise 3 CDRs, for example in a heavy chain variable region according to SEQ ID NO: 38 and/or may comprise 3 CDRs in a light chain variable region according to SEQ ID NO:42.

Suitably the reporter molecule which binds to reduced-IL33-binding molecule complexes comprises 3 CDRs in a heavy chain variable region (VH) wherein the VH has an amino acid sequence at least 85%, at least 90%, for example 91, 92, 93, 94, 95, 96, 97, 98, 99 or 100% identical to a VH according to SEQ ID NO: 38, and/or comprises 3 CDRs in a light chain variable region (VL) wherein the VL has an amino acid sequence at least 85%, at least 90%, for example 91, 92, 93, 94, 95, 96, 97, 98, 99 or 100% identical to a VL according to SEQ ID NO: 42.

Suitably the reporter molecule which binds to reduced-IL33-binding molecule complexes may comprise a variable heavy domain (VH) and a variable light domain (VL) having VHCDRs 1-3 of: SEQ ID NO: 35, 36 and 37 wherein one or more VHCDRs have 3 or fewer single amino acid substitutions, deletions or insertions.

Suitably, one or more of the VHCDRs comprise 3, 2, 1, or no single amino acid substitutions, deletions or insertions. Suitably the VHCDRs consist of SEQ ID NO: 35, 36 and 37.

Suitably the reporter molecule which binds to reduced-IL33-binding molecule complexes may comprise a variable heavy domain (VH) and a variable light domain (VL) having VLCDRs 1-3 of: SEQ ID NO: 39, 40 and 41 wherein one or more VLCDRs have 3 or fewer single amino acid substitutions, deletions or insertions.

Suitably, one or more of the VLCDRs comprise 3, 2, 1, or no single amino acid substitutions, deletions or insertions. Suitably the VLCDRs consist of SEQ ID NO: 39, 40 and 41.

Suitably the reporter molecule which binds to reduced-IL33-binding molecule complexes may comprise a VH having VHCDRs 1-3 of: SEQ ID NO: 35, 36 and 37, and a VL having VLCDRs of SEQ ID NO: 39, 40 and 41, wherein one or more of the CDRs have 3 or fewer single amino acid substitutions, deletions or insertions. Suitably, one or more of the CDRs have 3, 2, 1, or no single amino acid substitutions, deletions or insertions. Suitably such substitutions may be conservative substitutions.

Suitably the reporter molecule which binds to reduced-IL33-binding molecule complexes may comprise a VH having VHCDRs 1-3 of: SEQ ID NO: 35, 36 and 37 and a VL having VLCDRs of SEQ ID NO: 39, 40 and 41. Suitably the reporter molecule which binds to reduced-IL33-binding molecule complexes may comprise a VH having VHCDRs 1-3 consisting of: SEQ ID NO: 35, 36 and 37, and a VL having VLCDRs consisting of SEQ ID NO: 39, 40 and 41.

Suitably the reporter molecule which binds to reduced-IL33-binding molecule complexes may comprise any of the following CDRs: a VHCDR1 having the sequence of: SEQ ID NO:35, a VHCDR2 having the sequence of: SEQ ID NO:36, a VHCDR3 having the sequence of: SEQ ID NO: 37, a VLCDR1 having the sequence of: SEQ ID NO:39, a VLCDR2 having the sequence of: SEQ ID NO:40, and a VLCDR3 having the sequence of: SEQ ID NO:41.

Suitably the reporter molecule which binds to reduced-IL33-binding molecule complexes may comprise a VHCDR1 having the sequence of: SEQ ID NO: 35 a VHCDR2 having the sequence of: SEQ ID NO: 36 a VHCDR3 having the sequence of: SEQ ID NO: 37 a VLCDR1 having the sequence of: SEQ ID NO: 39 a VLCDR2 having the sequence of: SEQ ID NO: 40 and a VLCDR3 having the sequence of: SEQ ID NO: 41.

Suitably therefore, the reporter molecule which binds to reduced-IL33-binding molecule complexes is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO:38.

Suitably, therefore the reporter molecule comprises a VH, wherein a VH disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the reporter molecule which binds to reduced-IL33-binding molecule complexes is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 28, and comprises VHCDRs 1-3 of: SEQ ID NO: 35, 36 and 37, wherein one or more VHCDRs have 3 or fewer single amino acid substitutions, deletions or insertions. Suitably, one or more VHCDRs have 3, 2, 1, or no single amino acid substitutions, deletions or insertions.

Suitably therefore, the reporter molecule which binds to reduced-IL33-binding molecule complexes is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 38, and comprises VHCDRs 1-3 of: SEQ ID NO: 35, 36 and 37.

Suitably therefore, the reporter molecule which binds to reduced-IL33-binding molecule complexes is an antibody or binding fragment thereof comprising a VH and VL, wherein the VL has an amino acid sequence at least 90%, for example 91, 92, 93, 94, 95, 96, 97, 98, 99 or 100% identical to a VL according to SEQ ID NO:42.

Suitably, therefore the reporter molecule comprises a VL, wherein a VL disclosed above, has a sequence with 1, 2, 3 or 4 amino acids in the framework deleted, inserted and/or independently replaced with a different amino acid i.e, substituted. Suitably such substitutions may be conservative substitutions.

Suitably therefore, the reporter molecule which binds to reduced-IL33-binding molecule complexes is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 32, and comprises VLCDRs 1-3 of: SEQ ID NO: 39, 40 and 41 wherein one or more VLCDRs have 3 or fewer single amino acid substitutions, deletions or insertions. Suitably, one or more VLCDRs have 3, 2, 1, or no single amino acid substitutions, deletions or insertions.

Suitably therefore, the reporter molecule which binds to reduced-IL33-binding molecule complexes is an antibody or binding fragment thereof comprising a VH and VL, wherein the VL has an amino acid sequence at least 85%, at least 90%, for example 91, 92, 93, 94, 95, 96, 97, 98, 99 or 100% identical to a VL according to SEQ ID NO: 42, and comprises VLCDRs 1-3 of: SEQ ID NO:39, 40 and 41.

Suitably therefore, the reporter molecule which binds to reduced-IL33-binding molecule complexes is an antibody or binding fragment thereof comprising a VH and VL, wherein the VH has an amino acid sequence at least 85%, at least 90%, for example 91, 92, 93, 94, 95, 96, 97, 98, 99 or 100% identical to a VH according to SEQ ID NO: 38, and comprises VHCDRs 1-3 of: SEQ ID NO: 35, 36 and 37, and wherein the VL has an amino acid sequence at least 85%, at least 90%, for example 91, 92, 93, 94, 95, 96, 97, 98, 99 or 100% identical to a VL according to SEQ ID NO: 42, and comprises VLCDRs 1-3 of: SEQ ID NO: 39, 40 and 41.

Suitably therefore, the reporter molecule which binds to reduced-IL33-binding molecule complexes is an antibody or binding fragment thereof comprising a VH, wherein the VH comprises an amino acid sequence according to SEQ ID NO: 38. Suitably therefore, the reporter molecule which binds to reduced-IL33-binding molecule complexes is an antibody or binding fragment thereof comprising a VL, wherein the VL comprises an amino acid sequence according to SEQ ID NO: 42.

Suitably therefore, the reporter molecule which binds to reduced-IL33-binding molecule complexes is an antibody or binding fragment thereof comprising a VH and a VL, wherein the VH has an amino acid sequence consisting of SEQ ID NO: 38, and the VL has an amino acid sequence consisting of SEQ ID NO: 42.

### Oxidised IL-33 Reporter Molecule

Suitably the reporter molecule which binds to oxidised-IL33-binding molecule complexes may be obtained from any suitable supplier. For example, suitably the reporter molecule which binds to oxidised-IL33-binding molecule complexes is AF3625 from R&D systems.

Optionally, however, the oxidised IL-33 binding molecule described herein may also be used as an oxidised IL-33 reporter molecule. Suitably, the binding molecule of (b) (ii) of the first aspect or the binding molecule of the fifth aspect may also be used as a reporter molecule. Suitably therefore, in some assays for detecting oxidised IL-33, the binding molecule and the reporter molecule may be the same. Suitably therefore, in some assays for detecting oxidised IL-33, the binding molecule and the reporter molecule may both be the molecule as defined in step (b) (ii) of the first aspect or the fifth aspect. Suitable details of the oxidised IL-33 binding molecule which may be used as a reporter molecule are described hereinabove under the heading ‘Oxidised IL-33 binding molecule-AB1070141’.

Suitably therefore, there is provided a reporter molecule which binds to oxidised-IL33-binding molecule complexes comprising a variable heavy domain having VHCDRs 1-3 of SEQ ID NOS: 9, 10 and 11, respectively, and a variable light domain having VLCDRs 1-3 of SEQ ID NOS: 13, 14 and 15, respectively, wherein one or more CDRs have 3 or fewer single amino acid substitutions, deletions or insertions. Suitable further sequences defining such a reporter molecule are described elsewhere herein in relation to molecule AB1070141. In one instance, the or each reporter molecule may comprise a detectable label. In one instance, the or each reporter molecule is conjugated to a detectable label.

### IL-33/sST2 Reporter Molecule

Suitably the reporter molecule which binds to IL-33/sST2-binding molecule complexes may be obtained from any suitable supplier. For example, suitably the reporter molecule which binds to IL-33/sST2-binding molecule complexes is MAB5232 from R&D systems.

Optionally, however, the IL-33/sST2 binding molecule described herein may also be used as an IL-33/sST2 reporter molecule. Suitably, the binding molecule of (b) (iii) of the first aspect or the binding molecule of the sixth aspect may also be used as a reporter molecule. Suitably therefore, in some assays for detecting IL-33/sST2, the binding molecule and the reporter molecule may be the same. Suitably therefore, in some assays for detecting IL-33/sST2, the binding molecule and the reporter molecule may both be the molecule as defined in step (b) (iii) of the first aspect or the sixth aspect. Suitable details of the IL-33/sST2 binding molecule which may be used as a reporter molecule are described hereinabove under the heading ‘IL33/sST2 Binding Molecule-AB1070008’.

Suitably therefore, there is provided a reporter molecule which binds to IL-33/sST2-binding molecule complexes comprising a variable heavy domain having VHCDRs 1-3 of: SEQ ID NOs: 17, 18 and 19, respectively, and a variable light domain having VLCDRs 1-3 of SEQ ID NOs: 21, 22 and 23, respectively, wherein one or more CDRs have 3 or fewer single amino acid substitutions, deletions or insertions. Suitable further sequences defining such a reporter molecule are described elsewhere herein in relation to molecule AB1070008. In one instance, the or each reporter molecule may comprise a detectable label. In one instance, the or each reporter molecule is conjugated to a detectable label.

### Polynucleotides Encoding the Binding and Reporter Molecules

The present disclosure also provides for polynucleotide molecules encoding binding molecules and reporter molecules of the invention. Suitably isolated polynucleotide molecules encoding binding molecules and reporter molecules of the invention.

Suitably, the isolated polynucleotide comprises, consists essentially of, or consists of a nucleic acid encoding an immunoglobulin heavy chain variable domain (VH domain), where the sequences of the CDRs of the VH domain are: a VHCDR1 sequence comprising the amino acid sequence selected from SEQ ID NO: 1, 9, 17,25 and 35, a VHCDR2 sequence comprising the amino acid sequence selected from SEQ ID NO: 2, 10, 18, 26 and 36, a VHCDR3 sequence comprising the amino acid sequence selected from SEQ ID NO: 3, 11, 19, 27 and 37.

Suitably, the isolated polynucleotide comprises, consists essentially of, or consists of a nucleic acid encoding an immunoglobulin heavy chain variable domain (VH domain) having an amino acid sequence that is at least 85%, at least 90%, at least 95%, for example 96, 97, 98, 99 or 100% identical to a sequence selected from SEQ ID NO: 4, 12, 20, 28 and 38.

Suitably, the isolated polynucleotide comprises, consists essentially of, or consists of a nucleic acid encoding an immunoglobulin light chain variable domain (VL domain), where the sequences of the CDRs of the VL domain are: a VLCDR1 sequence comprising the amino acid sequence selected from SEQ ID NO: 5, 13, 21, 29 and 39, a VLCDR2 sequence comprising the amino acid sequence selected from SEQ ID NO: 6, 14, 22, 30 and 40, a VLCDR3 sequence comprising the amino acid sequence selected from SEQ ID NO: 7, 15, 23, 31 and 41.

Suitably, the isolated polynucleotide comprises, consists essentially of, or consists of a nucleic acid encoding an immunoglobulin light chain variable domain (VL domain) having an amino acid sequence that is at least 85%, at least 90%, at least 95%, for example 96, 97, 98, 99 or 100% identical to a sequence selected from SEQ ID NO: 8, 16, 24, 32 and 42.

Any of the polynucleotides described above may further include additional nucleic acids, encoding, e.g., a signal peptide to direct secretion of the encoded polypeptide, antibody constant regions as described herein, or other heterologous polypeptides as described herein. Also, as described in more detail elsewhere herein, the present disclosure includes compositions comprising one or more of the polynucleotides described above.

In one embodiment, there is provided a composition comprising a first polynucleotide and second polynucleotide wherein said first polynucleotide encodes a VH domain as described herein and wherein said second polynucleotide encodes a VL domain as described herein.

The present disclosure also includes fragments of the polynucleotides of the disclosure, as described elsewhere. Additionally polynucleotides that encode Fab fragments, and other derivatives, as described herein, are also contemplated by the disclosure.

The polynucleotides may be produced or manufactured by any method known in the art. For example, if the nucleotide sequence of the binding molecule is known, a polynucleotide encoding the binding molecule may be assembled from chemically synthesized oligonucleotides (e.g., as described in Kutmeier et al., Bio Techniques 17:242 (1994)), which, briefly, involves the synthesis of overlapping oligonucleotides containing portions of the sequence encoding the binding molecule, annealing and ligating of those oligonucleotides, and then amplification of the ligated oligonucleotides by PCR.

Alternatively, a polynucleotide encoding a binding molecule or reporter molecule, such as an antibody, or antigen-binding fragment, variant, or derivative thereof of the disclosure, may be generated from nucleic acid from a suitable source. If a clone containing a nucleic acid encoding a particular antibody is not available, but the sequence of the antibody molecule is known, a nucleic acid encoding the antibody may be chemically synthesized or obtained from a suitable source (e.g., an antibody cDNA library, or a cDNA library generated from, or nucleic acid, preferably poly A+RNA, isolated from, any tissue or cells expressing the antibody or other anti-IL-33 antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or other anti-IL-33 antibody. Amplified nucleic acids generated by PCR may then be cloned into replicable cloning vectors using any method well known in the art.

Once the nucleotide sequence and corresponding amino acid sequence of the binding molecule or reporter molecule is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al. (1990) Molecular Cloning, A Laboratory Manual (2nd ed.; Cold Spring Harbor Laboratory, Cold Spring Harbor, N.Y.) and Ausubel et al., eds. (1998) Current Protocols in Molecular Biology (John Wiley & Sons, NY), which are both incorporated by reference herein in their entireties), to generate binding molecules having a different amino acid sequence, for example to create amino acid substitutions, deletions, and/or insertions.

A polynucleotide encoding a binding molecule or reporter molecule, e.g., an antibody, or antigen-binding fragment, variant, or derivative thereof, can be composed of any polyribonucleotide or polydeoxyribonucleotide, which may be unmodified RNA or DNA or modified RNA or DNA. For example, a polynucleotide encoding an antibody, or antigen-binding fragment, variant, or derivative thereof can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding a binding molecule, e.g., an antibody, or antigen-binding fragment, variant, or derivative thereof can be composed of triple-stranded regions comprising RNA or DNA or both RNA and DNA. A polynucleotide encoding a binding molecule, e.g., antibody, or antigen-binding fragment, variant, or derivative thereof, may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

### Expression of the Binding and Reporter Molecules

Polynucleotides encoding a binding molecule or reporter molecule of the invention are typically inserted in an expression vector for introduction into host cells that may be used to produce the desired quantity of the binding molecule or reporter molecule. As such, an expression vector comprising one or more polynucleotides encoding a binding molecule or reporter molecule of the invention, and a host cell comprising said expression vector are encompassed in the present invention.

Recombinant expression of a binding molecule or reporter molecule, e.g., a heavy and/or light chain of an antibody that binds to a target molecule described herein, e.g., reduced IL-33, oxidised IL-33 or IL-33/sST2, requires construction of an expression vector containing a polynucleotide that encodes the binding molecule or reporter molecule. Once a polynucleotide encoding the binding molecule or reporter molecule, for example a heavy and/or light chain of an antibody, or portion thereof (preferably containing the heavy or light chain variable domain), of the disclosure has been obtained, the vector for the production of the molecule may be produced by recombinant DNA technology using techniques well known in the art.

DNA sequences that encode the binding molecule or reporter molecule may be made, either simultaneously or separately, using reverse transcriptase and DNA polymerase in accordance with well-known methods. PCR may be initiated by consensus constant region primers or by more specific primers based on the published DNA and amino acid sequences. PCR also may be used to isolate DNA clones encoding antibody light and heavy chains. In this case the libraries may be screened by consensus primers or larger homologous probes, such as mouse constant region probes.

Thus, methods for preparing a protein by expressing a polynucleotide containing a binding molecule or reporter molecule encoding nucleotide sequence are described herein. Methods that are well known to those skilled in the art can be used to construct expression vectors containing binding molecule coding sequences or reporter molecule coding sequences and appropriate transcriptional and translational control signals. These methods include, for example, in vitro recombinant DNA techniques, synthetic techniques, and in vivo genetic recombination. The disclosure, thus, provides replicable vectors comprising a nucleotide sequence encoding a binding molecule or reporter molecule of the disclosure, for example a heavy or light chain, or a heavy or light chain variable domain, operably linked to a promoter. Such vectors may include the nucleotide sequence encoding the constant region of an antibody molecule (see, e.g., PCT Publication WO 86/05807; PCT Publication WO 89/01036; and U.S. Pat. No. 5,122,464) and the variable domain of an antibody may be cloned into such a vector for expression of the entire heavy and/or light chain.

The term “vector” or “expression vector” is used herein to mean vectors used in accordance with the present disclosure as a vehicle for introducing into and expressing a desired polynucleotide in a host cell. As known to those skilled in the art, such vectors may easily be selected from the group consisting of plasmids, phages, viruses and retroviruses. In general, vectors compatible with the instant disclosure will comprise a selection marker, appropriate restriction sites to facilitate cloning of the desired gene and the ability to enter and/or replicate in eukaryotic or prokaryotic cells.

For the purposes of this disclosure, numerous expression vector systems may be employed. For example, one class of vector utilizes DNA elements that are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells that have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals.

In one embodiment for expression of an antibody, the cloned variable region genes are inserted into an expression vector along with the heavy and light chain constant region genes (preferably human) synthesized as discussed above. Of course, any expression vector that is capable of eliciting expression in eukaryotic cells may be used in the present disclosure. Examples of suitable vectors include, but are not limited to plasmids pcDNA3, pHCMV/Zeo, pCR3.1, pEF 1/His, pIND/GS, pRc/HCMV2, pSV40/Zeo2, pTRACER-HCMV, PUB6/V5-His, pVAXI, and pZeoSV2 (available from Invitrogen, San Diego, Calif.), and plasmid pCI (available from Promega, Madison, Wis.). In general, screening large numbers of transformed cells for those that express suitably high levels of immunoglobulin heavy and light chains is routine experimentation that can be carried out, for example, by robotic systems.

More generally, once the vector or DNA sequence encoding the binding molecule or reporter molecule has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway (1988) “Mammalian Expression Vectors” in Vectors, ed. Rodriguez and Denhardt (Butterworths, Boston, Mass.), Chapter 24.2, pp. 470-472. Typically, plasmid introduction into the host is via electroporation. The host cells harbouring the expression construct are grown under conditions appropriate to the production of the binding molecule or reporter molecule, and assayed for protein synthesis. Exemplary assay techniques include enzyme-linked immunosorbent assay (ELISA), radioimmunoassay (RIA), or fluorescence-activated cell sorter analysis (FACS), immunohistochemistry and the like.

The expression vector is transferred to a host cell by conventional techniques, and the transfected cells are then cultured by conventional techniques to produce a binding molecule or reporter molecule for use in the methods described herein. Thus, the disclosure includes host cells containing a polynucleotide encoding a binding molecule or reporter molecule of the disclosure, for example a heavy and/or light chain, or a variable heavy and/or variable light chain, operably linked to a heterologous promoter.

In one embodiment, there is provided a culture medium comprising a host cell of the invention. In one embodiment there is provided a fermentation vessel comprising said culture medium.

Suitably the culture medium and the fermentation vessel are suitable for carrying out the method of producing a binding molecule or reporter molecule of the invention.

As used herein, “host cells” refers to cells that harbour vectors constructed using recombinant DNA techniques and encoding at least one heterologous polynucleotide. In descriptions of processes for isolation of binding molecules or reporter molecules from recombinant hosts, the terms “cell” and “cell culture” are used interchangeably to denote the source of the binding molecule or reporter molecule unless it is clearly specified otherwise. In other words, recovery of polypeptide from the “cells” may mean either from spun down whole cells, or from the cell culture containing both the medium and the suspended cells.

A variety of host-expression vector systems may be utilised to express binding molecules or reporter molecules for use in the methods described herein. Such host-expression systems represent vehicles by which the coding sequences of interest may be produced and subsequently purified, but also represent cells that may, when transformed or transfected with the appropriate nucleotide coding sequences, express a molecule of the disclosure in situ. These include, but are not limited to, microorganisms such as bacteria (e.g., E. coli, B. subtilis) transformed with recombinant bacteriophage DNA, plasmid DNA or cosmid DNA expression vectors containing coding sequences; yeast (e.g., Saccharomyces, Pichia) transformed with recombinant yeast expression vectors containing coding sequences; insect cell systems infected with recombinant virus expression vectors (e.g., baculovirus) containing coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing coding sequences; or mammalian cell systems (e.g., COS, CHO, BLK, 293, 3T3 cells) harbouring recombinant expression constructs containing promoters derived from the genome of mammalian cells (e.g., metallothionein promoter) or from mammalian viruses (e.g., the adenovirus late promoter; the vaccinia virus 7.5K promoter).

Preferably, bacterial cells such as Escherichia coli, and more preferably, eukaryotic cells, especially for the expression of whole recombinant antibody molecules, are used for the expression of a binding molecule or reporter molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al, Bio/Technology 8:2 (1990)).

The host cell line used for protein expression is often of mammalian origin; those skilled in the art are credited with ability to preferentially determine particular host cell lines that are best suited for the desired gene product to be expressed therein. Exemplary host cell lines include, but are not limited to, CHO (Chinese Hamster Ovary), DG44 and DUXB13 (Chinese Hamster Ovary lines, DHFR minus), HELA (human cervical carcinoma), CVI (monkey kidney line), COS (a derivative of CVI with SV40 T antigen), VERY, BHK (baby hamster kidney), MDCK, 293, WI38, R1610 (Chinese hamster fibroblast) BALBC/3T3 (mouse fibroblast), HAK (hamster kidney line), SP2/0 (mouse myeloma), P3. times.63-Ag3.653 (mouse myeloma), BFA-IcIBPT (bovine endothelial cells), RAJI (human lymphocyte) and 293 (human kidney). Host cell lines are typically available from commercial services, the American Tissue Culture Collection or from published literature.

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used.

For long-term, high-yield production of recombinant proteins, stable expression is preferred. For example, cell lines that stably express the binding molecules or reporter molecules herein may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the binding molecule or reporter molecule.

A number of selection systems may be used, including, but not limited to, the herpes simplex virus thymidine kinase (Wigler et al., Cell 13:223 (1977)), hypoxanthine-guanine phosphoribosyltransferase (Szybalska and Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1521 (1981)); gpt, which confers resistance to mycophenolic acid (Mulligan and Berg, Proc. Natl. Acad. Sci. USA 78:2012 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 52:573-596 (1993); Mulligan, Science 260:926-932 (1993); and Morgan and Anderson, Ann. Rev. Biochem. 62:191-217 (1993); TIB TECH 13 (5): 155-215 (May 1993); and hygro, which confers resistance to hygromycin (Santerre et al., Gene 30:141 (1984). Methods commonly known in the art of recombinant DNA technology which can be used are described in Ausubel et al. (1993) Current Protocols in Molecular Biology (John Wiley & Sons, NY); Kriegler (1990) “Gene Transfer and Expression” in A Laboratory Manual (Stockton Press, NY); Dracopoli et al. (eds) (1994) Current Protocols in Human Genetics (John Wiley & Sons, NY) Chapters 12 and 13; Colberre-Garapin et al. (1981) J. Mol. Biol. 150:1, which are incorporated by reference herein in their entireties.

The expression levels of a binding molecule or reporter molecule can be increased by vector amplification (for a review, see Bebbington and Hentschel (1987) “The Use of Vectors Based on Gene Amplification for the Expression of Cloned Genes in Mammalian Cells in DNA Cloning” (Academic Press, NY) Vol. 3. When a marker in the vector system expressing a binding molecule or reporter molecule is amplifiable, increase in the level of inhibitor present in culture of host cell will increase the number of copies of the marker gene. Since the amplified region is associated with the binding molecule gene, or reporter molecule gene, production of the antibody will also increase (Crouse et ah, Mol. Cell. Biol. 3:251 (1983)).

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-) affinity chromatography, e.g., after preferential biosynthesis of a synthetic hinge region polypeptide or prior to or subsequent to the HIC chromatography step described herein.

Genes encoding binding molecules or reporter molecules of the disclosure, or parts thereof such as the VH or VL regions, can also be expressed in non-mammalian cells such as insect, bacteria or yeast or plant cells. Bacteria that readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologous polypeptides must be isolated, purified and then assembled into functional molecules. Where tetravalent forms of antibodies are desired, the subunits will then self-assemble into tetravalent antibodies (WO 02/096948 A2).

In bacterial systems, a number of expression vectors may be advantageously selected, when a large quantity of such a protein is to be produced, vectors which direct the expression of high levels of fusion protein products that are readily purified may be desirable. Such vectors include, but are not limited, to the E. coli expression vector pUR278 (Ruther et al., EMBO J. 2:1791 (1983)), in which the coding sequence may be ligated individually into the vector in frame with the lacZ coding region so that a fusion protein is produced; pIN vectors (Inouye and Inouye, Nucleic Acids Res. iJ: 3101-3109 (1985); Van Heeke and Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pastons.

For expression in Saccharomyces, the plasmid YRp7, for example, (Stinchcomb et ah, Nature 282:39 (1979); Kingsman et al, Gene 7:141 (1979); Tschemper et al, Gene 10:151 (1980)) is commonly used. This plasmid already contains the TRP1 gene, which provides a selection marker for a mutant strain of yeast lacking the ability to grow in tryptophan, for example ATCC No. 44076 or PEP4-1 (Jones, Genetics 85:12 (1977)). The presence of the trpl lesion as a characteristic of the yeast host cell genome then provides an effective environment for detecting transformation by growth in the absence of tryptophan.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

Once a binding molecule or reporter molecule of the disclosure has been recombinantly expressed, it may be purified by any method known in the art for purification of an immunoglobulin molecule, for example, by chromatography {e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of antibodies of the disclosure is disclosed in U.S. Patent Application Publication No. 2002 0123057 A1.

### Kits

The present disclosure further provides a kit for detecting antigenically distinct forms of IL-33 in a biological sample.

Suitably the kit comprises at least one binding molecule and at least one reporter molecule. Suitably the binding molecule and reporter molecule are selected from any of those as defined hereinabove.

Suitably the kit comprises:
(i) An oxidised IL-33 binding molecule and a reporter molecule which binds to oxidised-IL33-binding molecule complexes; and/or
(ii) A reduced IL-33 binding molecule and a reporter molecule which binds to reduced-IL33-binding molecule complexes; and/or
(iii) An IL-33/sST2 binding molecule and a reporter molecule which binds to IL-33/sST2-binding molecule complexes.

Suitably the kit comprises (i) and (ii). Suitably the kit comprises (i) and (iii). Suitably the kit comprises (i) and (ii). Suitably the kit comprises (i), (ii) and (iii).

Suitably the reporter molecules are detectably labelled, suitably each comprises a label as described hereinabove.

Alternatively, in a further instance, there is provided an assay kit for detecting antigenically distinct forms of IL-33 in a biological sample comprising
(i) A binding molecule capable of binding to an antigenically distinct form of IL-33 selected from: reduced IL-33, oxidised IL-33 and/or IL-33/sST2 to form a complex.

Suitably, in such an instance, the kit comprises a reduced IL-33 binding molecule and/or an oxidised IL-33 binding molecule and/or an IL-33/sST2 binding molecule. Suitably, in such an instance, the kit comprises a reduced IL-33 binding molecule and an oxidised IL-33 binding molecule and an IL-33/sST2 binding molecule. Suitably, in such an instance, the or each binding molecule is detectably labelled.

Alternatively, the detectable label may be provided separately in the kit, suitably for attachment to the binding molecule or reporter molecule. Suitably therefore the kit may comprise a detectable label, suitable detectable labels are identified hereinabove

Suitably therefore, the kit further comprises a stimulating agent capable of stimulating or activating the detectable label of the or each binding molecule or reporter molecule.

Suitable biological samples for use with the kit are also defined hereinabove.

Suitably the kit is adapted for conducting an immunoassay as described hereinabove. Suitably the kit is adapted for conducting an ELISA or an ECLIA or a CLIA. Suitably the kit is adapted for conducting an S-plex assay. Suitably the kit is adapted for conducting an ECLIA such as an Elecsys® assay.

Suitably the kit may further comprise a solid support as described hereinabove. Suitably one or more of the binding molecules may be located on the solid support. Suitably the one or more binding molecules may be bound to the support or impregnated within the support. Suitably the solid support may be glass or a polymer, including, but not limited to cellulose, polyacrylamide, nylon, polystyrene, polyvinylchloride or polypropylene as explained hereinabove. Suitably the solid support may be in the form of tubes, beads, discs, microplates, columns, or any other surfaces suitable for conducting an assay method as explained hereinabove. In some embodiments, the kit may comprise beads or microparticles, suitably magnetic beads or microparticles.

Suitably the kit may further comprise a tag and capture partner. Suitably the kit may comprise biotin and streptavidin. In some embodiments, the kit may comprise streptavidin coated beads or microparticles, sutiably streptavidin coated magnetic beads or microparticles.

Optionally, the kit may also contain reagents such as buffers, washing fluid, distilled water, blocking agents, stop solutions and the like.

Suitably the kit may further comprise stimulating agents as already described herein. Suitably for stimulating the detectable label of the one or more reporter molecules or binding molecules. Suitable stimulating agents are molecules which stimulate a detectable label, which detectable labels may suitably be part of the one or more reporter molecules or binding molecules. Suitable stimulating agents may include substrates that can be acted upon by a detectable label. For example; an enzyme label to provide a detectable signal, or dyes that can react with a fluorophore label to provide a detectable signal. Suitable substrates to be used with a specific enzyme label are generally chosen for the production, upon hydrolysis by the corresponding enzyme, of a detectable colour change. For example, p-nitrophenyl phosphate is suitable for use with alkaline phosphatase conjugates; for peroxidase conjugates, 1,2-phenylenediamine or toluidine are commonly used. Suitably fluorogenic substrates may be used, which yield a fluorescent product. Suitably the stimulating agent may be a reactant, such as an electron donor. Suitably an amine compound, such as tripropylamine, or dibutyl ethanolamine

Alternatively the kit may further comprise one or more labels for detecting the one or more reporter molecules or binding molecules. Suitably the labels are detectable. Suitably the one or more labels specifically bind to the one or more reporter molecules or binding molecules. Suitable labels are discussed herein above. Suitably the kit may comprise: an oxidised IL-33 complex detectable label, and/or a reduced IL-33 complex detectable label and/or an IL-33/sST2 complex detectable label.

Suitably the kit may further comprise means for conducting the assay. Means for conducting the assay may suitably comprise a container such as a test tube, an Eppendorf® tube, a plate, and the like. Suitably if the kit comprises a solid support, the solid support may also act as the container for conducting the assay. Suitably the kit comprises one or more microwell plates. Suitably the kit comprises one or more microwell plates upon which the one or more binding molecules are immobilised.

In one instance, there is further provided a support comprising one or more binding molecules immobilised thereon. Suitably the one or more binding molecules are as defined according to the fourth, fifth or sixth aspects of the disclosure.

Suitably the kit may further comprise separating means. Suitably the separating means is for purifying the sample and/or isolating the one or more antigenically distinct forms of IL-33 from the sample. Suitable means for purifying or isolating may comprise filters, columns, beads, etc.

Suitably the kit may further comprise instructions, suitably for directing the user on how to carry out an assay using the kit Suitably for directing a user on how to carry out a method of the disclosure.

In one embodiment, the kit comprises:
(i) An oxidised IL-33 binding molecule and a reporter molecule which binds to oxidised-IL33-binding molecule complexes; and/or
(ii) A reduced IL-33 binding molecule and a reporter molecule which binds to reduced-IL33-binding molecule complexes; and/or
(iii) An IL-33/sST2 binding molecule and a reporter molecule which binds to IL-33/sST2-binding molecule complexes, wherein each binding molecule comprises a biotin tag, and wherein each reporter molecule comprises a chemiluminescent label;
(iv) A streptavidin coated magnetic bead;
(v) A reactant capable of activating the chemiluminescent label in the presence of an electric current/voltage

Suitably wherein the chemiluminescent label is a ruthenium compound and the reactant is tripropylamine, or dibutyl ethanolamine.

Suitably the kit may further comprise a magnetic electrode

The disclosure will now be described with reference to the following non-limiting examples, in which:

### Examples

### Hybridoma Campaign for Generation of Binding Molecules and Reporter Molecules

Monoclonal antibodies were produced via hybridoma technology from wildtype outbred mice. Antibodies AB1070019 and AB1070141 were isolated from mice immunised with human IL-33 and antibodies AB1070008, AB1070069 and AB1070012 from mice immunised with human IL-33 complexed with MEDI3506 in Fab format.
AB1070012 (mouse IgG1)
AB1070019 (mouse IgG2b)
AB1070141 (mouse IgG1)
AB1070008 (mouse IgG1)

A repetitive immunisation at multiple sites (RIMS) strategy was used, in which CD1 mice were immunised as follows:
Minus 4 days: pre-bleed
0 days: prime immunisation
7 days: second boost
13 days: first bleed
15 days: third boost
20 days: second bleed
22 days: fourth boost
24 days: fifth boost
28 days: terminal bleed and spleen and lymph node harvest

For the immunisation, twelve mice were divided into 2 groups, each group containing 6 animals. Animals were immunised with human recombinant IL-33 or human recombinant IL-33 in complex with MEDI3506 fab.

Lymphoid cells from the immunised mice were harvested and mixed with Sp2/0 Ag14 myeloma cells, and hybridomas were formed using electrofusion. Following fusion, cells were resuspended in semi-solid media containing the selection reagent Hypoxanthine/Azaserine and an anti-mouse IgG FITC conjugated antibody.

Hybridoma clones were grown for 13-17 days, then FITC positive, IgG expressing clones were picked from semi-solid media using the ClonePixFL robot into liquid culture media. After 3-7 days of growth, IgG containing supernatants were screened for binding using Homogeneous

Time Resolved Fluorescence (HTRF) binding assays. Positive hybridomas were overgrown in 24 well plates, supernatants purified using ProPlus Phynexus tips, and binding properties were confirmed. cDNA from selected hybridomas was prepared and the antibody variable heavy (VH) and light (VL) chain sequences determined. The VH domains were cloned into expression vectors containing the mouse heavy constant domains equivalent to the original hybridoma cell line, i.e. mouse IgG1 except for AB1070019, which was mouse IgG2b. The VL domains were cloned into expression vectors containing mouse kappa constant domains. To generate purified recombinant IgGs, the heavy and light chain IgG expression vectors were transiently transfected into CHO-cells. IgGs were purified from culture supernatant using MabSelect Sure columns on an AKTAxpress. The amino acid sequences for each of the antibody variable domains is shown below.

TABLE 1
Affinity measurements (BIAcore) for hybridoma-derived mAb

| | IL-33 form | | | | | | | | |
|---|---|---|---|---|---|---|---|---|---|
| | Reduced IL-33 | | | Oxidized IL-33 | | | IL-33/sST2 complex | | |
| | mAb | | | | | | | | |
| | K_{d} | Kₒₙ | K_{off} | K_{d} | Kₒₙ | K_{off} | K_{d} | Kₒₙ | K_{off} |
| | (pM) | (1/MS) | (1/S) | (pM) | (1/MS) | (1/S) | (pM) | (1/MS) | (1/S) |
| AB1070012 | 59 | 2.7e6 | 1.59e−4 | | | | | | |
| AB1070019 | 68 | 2.35e6 | 1.61e−4 | | | | | | |
| AB1070141 | | | | 1830 | 1.86e5 | 3.4e−4 | | | |
| AB1070008 | 895 | 5.7e5 | 5.14e−4 | | | | 26500 | 1.3e4 | 3.43e−4 |

### Development of an Assay for Detection of Different IL-33 Forms

### Reduced IL-33

Briefly recombinant reduced IL-33 (112-270 amino acids) was expressed in E. coli and purified to homogeneity (as described in WO216/156440 A1 and Cohen et al., 2015).

### Oxidised IL-33

Oxidised IL-33 (oxIL-33) was generated from a stock of N-terminally His-Avi-tagged, reduced IL-33. Reduced IL-33 was oxidised by dilution to a final concentration of 0.5 mg/ml in 60% IMDM medium (with no phenol red), 40% DPBS and incubation at 37° C. overnight. Aggregates generated during the oxidation process were removed from the sample by loading it on a HiTrap Q HP anion exchange column-under the loading conditions, aggregates bound to the column and monomeric oxIL-33 flowed through and was collected. Tags were cleaved from the oxIL-33 by incubation with Factor Xa at a final concentration of 10 U/mg of oxIL-33 for 120 min at room temperature. To deplete the sample of any remaining reduced IL-33, soluble human ST2 extracellular domain fused to human IgG1 Fc was incubated with the sample and bound the reduced IL-33. The sample was concentrated in a centrifugal concentrator with a 5,000 Da cut-off and loaded on a Superdex 75 26/600 column, which separated the monomeric oxIL-33 from the other components. Fractions containing oxIL-33 were pooled and concentrated and the final concentration of the sample was determined via UV absorbance spectroscopy (EC=14,690 M⁻¹ cm⁻¹).

IL-33/sST2 Complex hST2 extracellular domain sequence with a C-terminal Flag-His10 tag was expressed in suspension HEK Expi293F cells by transient transfection with the plasmid encoding the hST2Flag His10 sequence using Expifectamine. After 6-7 days cells were harvested by centrifugation and the supernatant cleared by filtration. Protein was purified from the cleared supernatant using affinity capture chromatography (HisTrap Excel). Eluted monomer fraction at MW around 38.7 kDa was pooled. Protein concentration was determined from its OD280 nm readout on Nanodrop with an extinction coefficient (units) of 1AU=0.77 mg/ml

IL-33/sST2 complexes were prepared by incubating reduced IL-33 and sST2-FH for 15 min at a molar ratio of 1:10.

### MSD Assays—Standard Curves

MSD standard bind plates were coated with 50 μl of 5 μg/ml capture Ab in PBS for ~18 h, washed in 300 μl of 3×PBS/0.05% Tween-20 (PBS-T), incubated in 200 μl of PBS/1% BSA for 1 h, and washed in 3×PBS-T. Recombinant human IL-33 standards were diluted in PBS-T containing 0.1% BSA and 25 μl added to wells and incubated for 2 h. Plates were washed in 3×PBS-T, incubated in 50 μl 1 μg/ml sulfo-tagged detection Ab for 2 h, washed 3×PBS-T, and incubated in 150 μl MSD 1×Read buffer for 10 min and read.

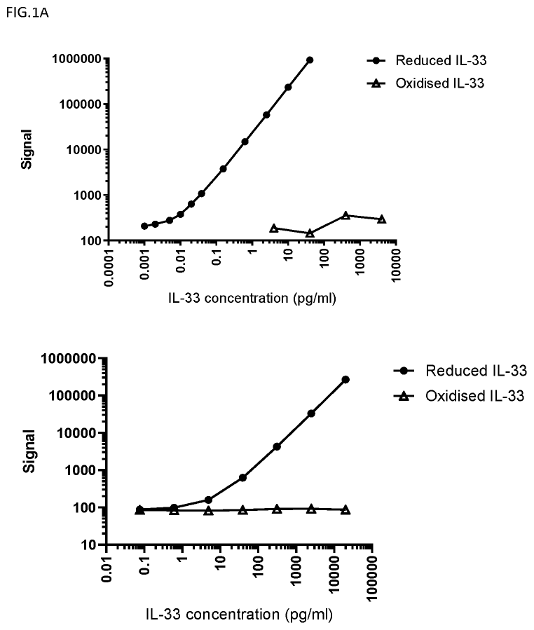

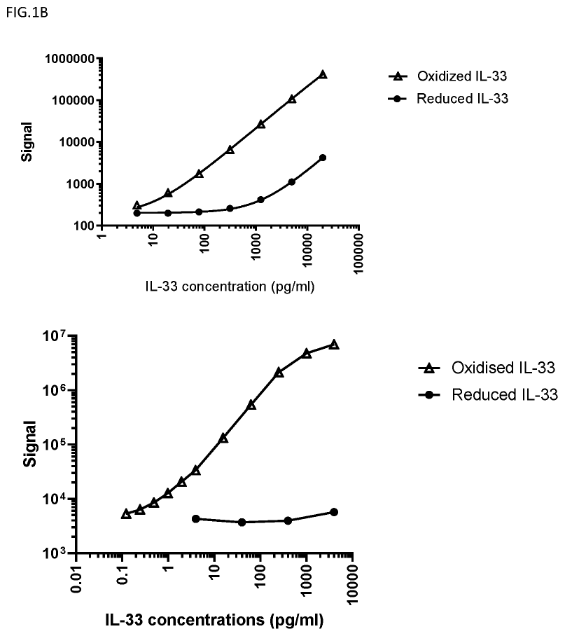

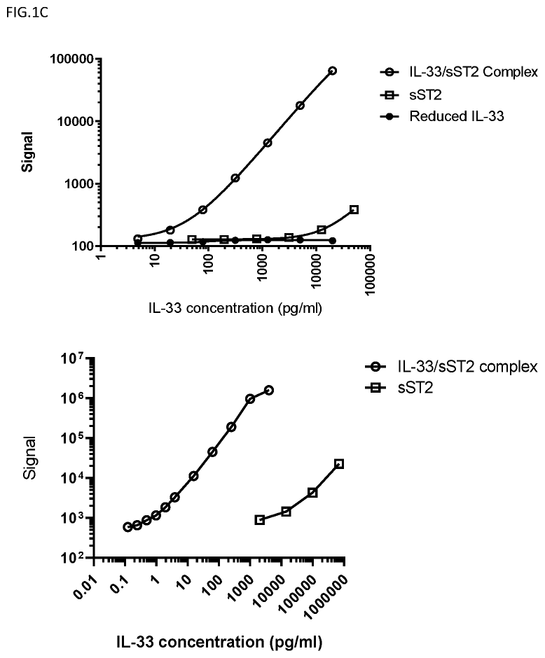

All standards and samples were run in duplicate. Incubation steps were carried out at room temperature on a shaking platform (450 rpm). All assays were read on an MSD Discovery Workbench Sector S600 reader. Graphical representations of concentration-dependent binding curves are shown in FIG. 1. For reduced IL-33 (FIG. 1A) and oxidised IL-33 (FIG. 1B), assay reagents showed no detectable binding to IL33/sST2 complex (data not shown). It was found that both AB1070012 and AB1070069 had equivalent performance for detecting reduced IL-33 (FIG. 5).

TABLE 2
Antibody pairs for IL-33 assays

| | Binding | Reporter |
|---|---|---|
| | Molecule | Molecule |
| Assay | (Capture Ab) | (Detection Ab) |
| Reduced IL-33 | AB1070012 | AB1070019 |
| Oxidized IL-33 | AB1070141 | R&D systems AF3625 |
| IL-33/sST2 complex | AB1070008 | R&D systems MAB5232 |
| Reduced IL-33 | AB1070069 | AB1070019 |

MSD S-PLEX Assays® for Reduced, oxIL-33 and IL-33/sST2 Complexes

MSD S-PLEX assays® were performed at MSD (Gaithersburg, USA) using S-PLEX® technology using the same Ab pairs for each form of IL-33 as described hereinabove for the standard MSD assays.

TABLE 3
Estimated lower limit of detection (LLOD) of
MSD assays and MSD S-PLEX assays ®

| | | MSD S-PLEX |
|---|---|---|
| | MSD assay | assay ® |
| IL-33 assay format | Estimated LLOD (pg/ml) | |
| Reduced IL-33 | 5.5 | 0.016 |
| Oxidized IL-33 | 12.6 | 0.030 |
| IL-33/sST2 complex | 14.2 | 0.370 |

ND-not determined

Estimated LLOD is calculated as concentration off the standard curve which produced signals which are 2.5 standard deviations above the diluent only (buffer only).

### MSD Assays—Biological Samples

All biological samples were stored aliquoted at −80C after collection

### Serum

Serum samples from healthy individuals, asthma patients (GINA stage 1-2) and COPD patients (GOLD stage1-2) were obtained from a commercial supplier (Seralabs/BioIVT, UK).

### Nasal Mucosal Lining Fluid

Mucosal lining fluid (MLF) was collected at baseline (−30, −15 min) and following a single timothy grass pollen (Aquagen, Denmark) nasal challenge (5-480 min) in atopic patients as described previously (Leaker et al., 2016). MLF was collected onto synthetic absorptive matrix (SAM, Hunts, UK) and eluted from SAM using 330 μl PBS/0.1% Tween-20/0.1% BSA.

### Normal Human Bronchial Epithelial Cell (NHBE) Culture Supernatants

NHBE cells (Cambrex, UK) were cultured as described previously (Scott et al., 2018). NHBE were plated overnight in 96-well plates (50K) and supernatants were collected from 1-1440 min following scratch wounding (Woundmaker, Essen Bioscience).

### Air-Liquid Interface (ALI) Culture Supernatants

ALI culture basolateral supernatants were collected 24 h after change of culture medium.

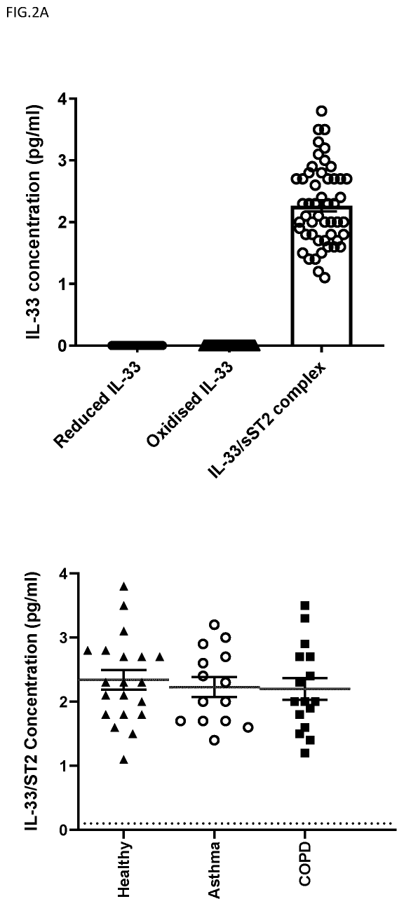

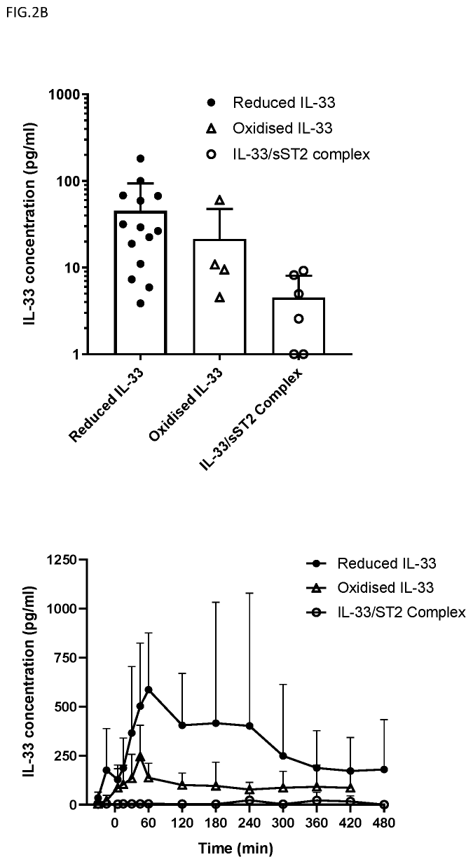

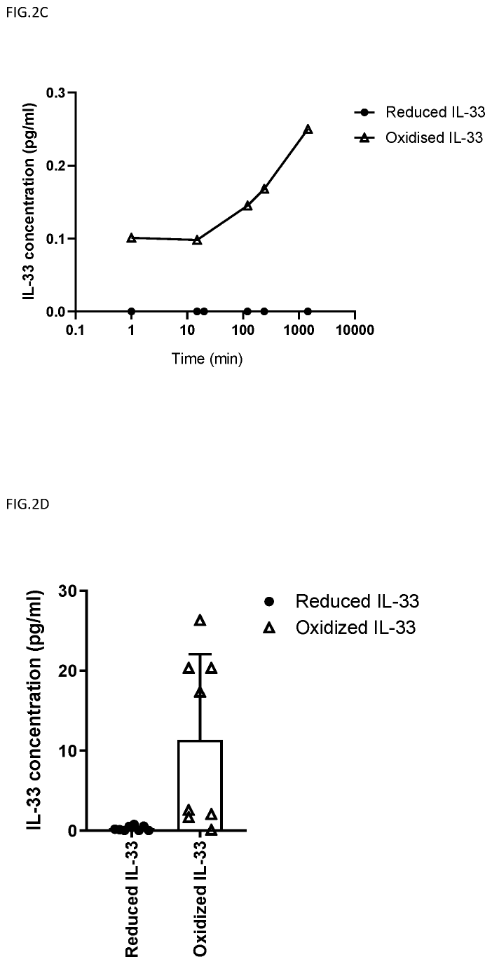

MSD standard bind plates were coated with 50 μl of 5 μg/ml capture Ab in PBS for ~18 h, washed in 300 μl of 3×PBS/0.05% Tween-20 (PBS-T), incubated in 200 μl of PBS/1% BSA for 1 h, and washed in 3×PBS-T. Biological samples were diluted in PBS-T containing 0.1% BSA (minimal dilution 1:2) and 25 μl added to wells and incubated for 2 h. Plates were washed in 3×PBS-T, incubated in 50 μl 1 μg/ml sulfo-tagged detection Ab for 2 h, washed 3×PBS-T, and incubated in 150 μl MSD 1×Read buffer for 10 min and read. Concentrations of IL-33 forms in biological samples were extrapolated from recombinant protein standard curves. The results show that the assays could be used to detect redIL-33, oxidised IL-33 and IL33/sST2 from a variety of alternative biological samples (FIG. 2A-2D).

### Use of the Assay to Detect IL-33/sST2 Complexes in Patients and Monitor Response to Treatment

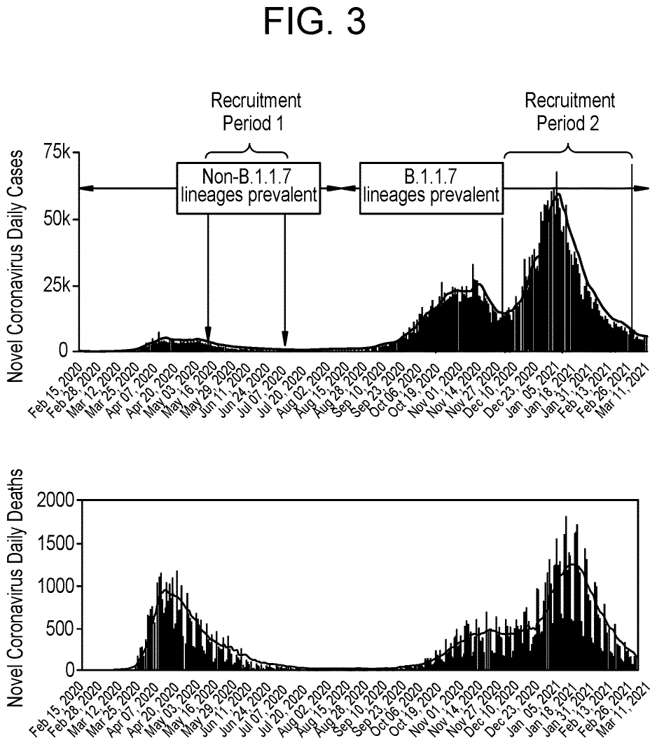

Serum samples from patients with SARS-COV-2 infection were obtained from patients enrolled in the Phase 2a ACCORD study (EudraCT Number: 2020-001736-95, Wilkinson et al., 2020). Patients were recruited during two time periods across the pandemic as shown in FIG. 3.

Patients were randomized and either received the standard of care (SoC) alone or in combination with an anti-IL-33 antibody (tozorakimab) treatment (300 mg IV and optional second dose if invasively ventilated at day 15) across a 29 day period. The 300 mg IV dose was administered by the following procedure: 2 ml of tozorakimab was diluted with 8 ml of saline to a total volume of 10 ml and administered to the patient via IV push over 1-2 minutes using IV-line filter and followed by IV flush with 5 ml of normal saline.

The SoC varied across the two periods of the ACCORD study as understanding of the SARS-CoV-2 virus developed. The SoC used during each period is shown below in Table 4. The SoC therapy in the second period of the ACCORD study more accurately reflects current best practice.

TABLE 4
SoC
ACCORD: Key SoC Therapies by Period

| | Period 1 | | Period 2 | |
|---|---|---|---|---|
| | (20 May 2020 to | | (8 Dec. 2020 to | |
| | 24 Jul. 2020) | | 2 Mar. 2021) | |
| | SoC | Tozorakimab | SoC | Tozorakimab |
| | (N = 12) | (N = 4) | (N = 32) | (N = 50) |
| Remdesivir | 17% | 25% | 56% | 36% |
| Dexamethasone | 58% | 75% | 100% | 94% |
| Tocilizumab | 0% | 0% | 16% | 12% |

Serum from patients dosed with tozorakimab+standard of care (n=46 as baseline, n=37 at day5, n=10 at dayl0) and SoC alone (n=37 baseline, n=23 at day 5, n=10 at day 10) were measured for IL-33/sST2 at 1:4 dilution.

All serum samples were stored aliquoted at −80C after collection.

MSD S-PLEX assays® were performed on the serum samples obtained from the patients at MSD (Gaithersburg, USA) using S-PLEX® technology using the antibody pair for IL-33/sST2 as described above to measure the levels of IL-33/sST2 in the serum.

The custom MSD S-PLEX IL-33/sST2 complex assay was prepared and validated by Mesoscale Discovery (MSD) and performed according to manufacturer's instructions. All incubations required plate shaking at room temperature (RT) unless otherwise stated. Plates were washed where stated with 3× with wash buffer (phosphate buffered saline (PBS)/0.05% Tween-20). Biotin coating capture mAb (Table 5) was diluted in Diluent 100 (MSD) with S-Plex coating reagent (MSD) to form a coating solution. Assay plates (MSD) were coated with 50 μl/well of coating solution and incubated for 1 h. Blocking reagent/solution was prepared by diluting S-Plex Blocker reagent (MSD) in Diluent 101 (MSD). Recombinant IL-33/sST2 complex standard curve was generated by diluting stock to the top standard concentration (Table 5) in Diluent 100 (MSD) and performing further 4-fold serial dilutions. 25 μl of blocking reagent was added to all wells and 25 μl of standards and samples were added to wells and incubated for 1.5 h. TURBO-boost solution was prepared by diluting TURBO-boost labelled detection mAb (Table 5) to working concentration in Diluent 3 (MSD). Plates were washed and 50 μl of TURBO-boost detection mAb solution added per well and incubated for 1 h. Plates were washed and 50 μl of enhancement solution (MSD) added/well and incubated for 30 min. Plates were washed and 50 μl of detection solution (MSD) added/well and incubated at 26° C. for 1 h. Plates washed and 150 μl of 1×Read Buffer A (MSD) added/well. Plates were read on an MSD MESO SECTOR S600 instrument.

TABLE 5
Concentrations of antibodies and IL-33/sST2 standard

| | IL-33/ST2 |
|---|---|
| | complex |
| Reagent | assay |
| Biotin coating capture Ab (μg/ml) | 0.25 |
| TURBO-boost detection Ab (μg/ml) | 0.1 |
| Top standard (Units/ml)* | 1000 |

*Data is expressed in Units/ml for IL-33/sST2 complex; this can be approximated to pg/ml if you assume 100% complex formation for IL-33/sST2 standard.

The MSD S-PLEX assay was sensitive enough to determine pg/ml levels of IL-33/sST2 in the serum of patients.

A subgroup analysis of the endpoint ‘Death or Respiratory Failure at Day 29’ was performed, based on median value for baseline IL-33/sST2. The median value at baseline was 30.15 U/ml and the subgroups were defined as IL-33 low: Baseline IL-33/sST2<30/15 U/ml; IL-33 high: Baseline IL-33/sST2>=30.15 U/ml.

The proportion of subjects who died by, or who were in respiratory failure at Day 29 was calculated for each treatment in each subgroup, and the relative risk (tozorakimab: placebo) was calculated for each subgroup together with 80% Confidence interval. Additionally, a logistic regression model was fitted for each subgroup, adjusting for age (continuous) and baseline severity (WHO score of ‘3 or 4’ versus ‘5’). From the logistic regression model, the odds ratio (tozorakimab: placebo) of being dead or in respiratory failure at Day 29 was calculated for each subgroup, with 80% confidence interval.

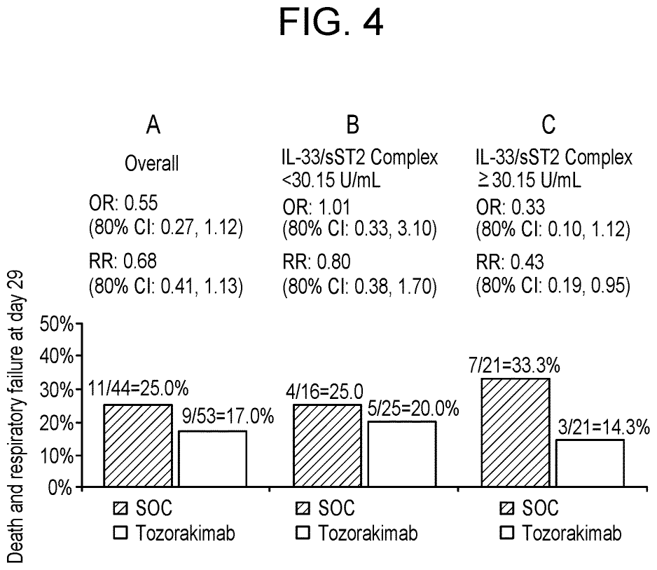

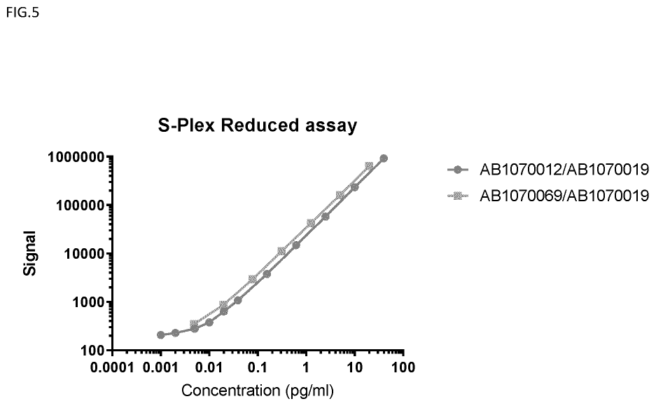

Using the assay, it was determined that those patients having a high baseline serum level of IL-33/sST2, of equal to or above 30.15 pg/ml, had a much reduced likelihood of death or respiratory failure after 29 days when treated with tozorakimab as shown in Table 6 and FIG. 4.

Such patients form a sub-group which are likely to respond well to IL-33 axis antagonist treatments, thereby providing a way of stratifying and selecting patients for treatment with IL-33 axis antagonists.

TABLE 6

| | | | SoC | tozorakimab + SoC |
|---|---|---|---|---|
| Sub-group | | | (n = 16) | (n = 25) |
| IL-33/sST2 | Death or | Yes: n(%) | 4 (25%) | 5 (20%) |
| <30.15 pg/ml | respiratory | | | |
| | failure at | No: n(%) | 12 (75%) | 20 (80%) |
| | day 29 | | | |
| | Risk ratio | | N/A | 0.8 (0.38, 1.70) |
| | (80% CI)* | | | |
| | Odds ratio | | N/A | 1.01 (0.33, 3.10) |
| | (80% CI)* | | | |
| | | | SoC | tozorakimab + SoC |
| | | | (n = 21) | (n = 21) |
| IL-33/sST2 | Death or | Yes: n(%) | 7 (33%) | 3 (14%) |
| ≥30.15 pg/ml | respiratory | | | |
| | failure at | No: n(%) | 14 (67%) | 18 (86%) |
| | day 29 | | | |
| | Risk ratio | | N/A | 0.43 (0.19, 0.95) |
| | (80% CI)* | | | |
| | Odds ratio | | N/A | 0.33 (0.10, 1.12) |
| | (80% CI)* | | | |

Sequences

| SEQ ID | | |
|---|---|---|
| Number | Key | Sequence |
| SEQ ID NO: | AB1070012 VH CDR1 | TYTMH |
| 1 | | |
| SEQ ID NO: | AB1070012 VH CDR2 | YINPYSGYTKYNQKFK |
| 2 | | |
| SEQ ID NO: | AB1070012 VH CDR3 | DSNFSYGGIYYYSMDY |
| 3 | | |
| SEQ ID NO: | AB1070012 VH | PVQLQQSGAELARPGASVKMSCKASGY |
| 4 | | TFTTYTMHWVKQRPGQGLEWIGYINPY |
| | | SGYTKYNQKFKDKATLTADKSSSTAYM |
| | | QLSSLTSEDSAVYYCARDSNFSYGGIYY |
| | | YSMDYWGQGTSVTVSS |
| SEQ ID NO: | AB1070012 VL CDR1 | KSSQSLLDSDGKTYLN |
| 5 | | |
| SEQ ID NO: | AB1070012 VL CDR2 | LVSKLDS |
| 6 | | |
| SEQ ID NO: | AB1070012 VL CDR3 | WQGTHFPRT |
| 7 | | |
| SEQ ID NO: | AB1070012 VL | DVVMTQTPLTLSVTIGQPASISCKSSQSL |
| 8 | | LDSDGKTYLNWLLQRPGQSPKRLIYLVS |
| | | KLDSGVPDRFTGSGSGTDFTLKISRVEA |
| | | EDLGVYYCWQGTHFPRTFGGGTKLEIK |
| SEQ ID NO: | AB1070141 VH CDR1 | DYWIE |
| 9 | | |
| SEQ ID NO: | AB1070141 VH CDR2 | EILPRSDSTNYNEKFK |
| 10 | | |
| SEQ ID NO: | AB1070141 VH CDR3 | HYHYDGSLFVY |
| 11 | | |
| SEQ ID NO: | AB1070141 VH | QVQLQQSGAELMKPGASVKLSCKATGY |
| 12 | | IVTDYWIEWVKQRPGHGLEWIGEILPRS |
| | | DSTNYNEKFKVRATFTADTSSNTAYMQ |
| | | LSSLTTEDSAIYYCASHYHYDGSLFVYW |
| | | GQGTLVTVSS |
| SEQ ID NO: | AB1070141 VL CDR1 | KSSQSLLNSRTRKNYL |
| 13 | | |
| SEQ ID NO: | AB1070141 VL CDR2 | WASTRES |
| 14 | | |
| SEQ ID NO: | AB1070141 VL CDR3 | KQSYSLPT |
| 15 | | |
| SEQ ID NO: | AB1070141 VL | DIVMSQSPSSLAVSAGEKVTMSCKSSQ |
| 16 | | SLLNSRTRKNYLAWYQQKPGQSPKLLIY |
| | | WASTRESGVPDRFTGSGSGTDFTLTISS |
| | | VQAEDLAVYYCKQSYSLPTFGGGTKLEI |
| | | K |
| SEQ ID NO: | AB1070008 VH CDR1 | SYYIH |
| 17 | | |
| SEQ ID NO: | AB1070008 VH CDR2 | WIYPGSGNTKYNEKFK |
| 18 | | |
| SEQ ID NO: | AB1070008 VH CDR3 | GFHYYGRMDY |
| 19 | | |
| SEQ ID NO: | AB1070008 VH | QVQLQQSGPELVKPGASVKTSCKASGY |
| 20 | | SFTSYYIHWVKQRPGQGLEWIGWIYPG |
| | | SGNTKYNEKFKGKATLTADTSSSTAFM |
| | | QLSSLTSEDSAVYYCASGFHYYGRMDY |
| | | WGQGTTLTVSS |
| SEQ ID NO: | AB1070008 VL CDR1 | RASQEISGYLS |
| 21 | | |
| SEQ ID NO: | AB1070008 VL CDR2 | STSTLDS |
| 22 | | |
| SEQ ID NO: | AB1070008 VL CDR3 | LQYASSPWT |
| 23 | | |
| SEQ ID NO: | AB1070008 VL | DIQMTQSPSSLSASLGERVSLTCRASQE |
| 24 | | ISGYLSWLQQKPDGTIKRLIYSTSTLDSG |
| | | VPKSFSGSRSGSDYSLTISSLESEDFAD |
| | | YYCLQYASSPWTFGGGTKLEIK |
| SEQ ID NO: | AB1070019 VH CDR1 | RYWMH |
| 25 | | |
| SEQ ID NO: | AB1070019 VH CDR2 | YINPYSGYTEYNQKFK |
| 26 | | |
| SEQ ID NO: | AB1070019 VH CDR3 | YSYYTYDGRGYAMDY |
| 27 | | |
| SEQ ID NO: | AB1070019 VH | QVQLQQSGAELAKPGASVKMSCKASG |
| 28 | | YTFTRYWMHWIKQRPGQGLEWIGYINP |
| | | YSGYTEYNQKFKDKATLTADKSSSTAY |
| | | MQLSSLTSEDSAVYYCARYSYYTYDGR |
| | | GYAMDYWGQGTSVTVSS |
| SEQ ID NO: | AB1070019 VL CDR1 | KASQNVGTNVA |
| 29 | | |
| SEQ ID NO: | AB1070019 VL CDR2 | SASYRYS |
| 30 | | |
| SEQ ID NO: | AB1070019 VL CDR3 | QQYNSYPFT |
| 31 | | |
| SEQ ID NO: | AB1070019 VL | DIVMTQSQKFMSTSVGDRVSVTCKASQ |
| 32 | | NVGTNVAWYQQKPGQSPKALIYSASYR |
| | | YSGVPDRFTGSGSGTDFTLTISNVQSED |
| | | LAEYFCQQYNSYPFTFGSGTKLEIK |
| SEQ ID NO: | IL33 | SITGISPITEYLASLSTYNDQSITFALEDE |
| 33 | (amino acids 112-270) | SYEIYVEDLKKDEKKDKVLLSYYESQHP |
| | | SNESGDGVDGKMLMVTLSPTKDFWLHA |
| | | NNKEHSVELHKCEKPLPDQAFFVLHNM |
| | | HSNCVSFECKTDPGVFIGVKDNHLALIK |
| | | VDSSENLCTENILFKLSET |
| SEQ ID NO: | ST2 (HIS-tagged) | KFSKQSWGLENEALIVRCPRQGKPSYT |
| 34 | | VDWYYSQTNKSIPTQERNRVFASGQLL |
| | | KFLPAAVADSGIYTCIVRSPTFNRTGYAN |
| | | VTIYKKQSDCNVPDYLMYSTVSGSEKNS |
| | | KIYCPTIDLYNWTAPLEWFKNCQALQGS |
| | | RYRAHKSFLVIDNVMTEDAGDYTCKFIH |
| | | NENGANYSVTATRSFTVKDEQGFSLFP |
| | | VIGAPAQNEIKEVEIGKNANLTCSACFGK |
| | | GTQFLAAVLWQLNGTKITDFGEPRIQQE |
| | | EGQNQSFSNGLACLDMVLRIADVKEED |
| | | LLLQYDCLALNLHGLRRHTVRLSRKNPI |
| | | DHHSDPAFLYKVVGAADYKDDDDKAAH |
| | | HHHHHHHHH |
| SEQ ID NO: | AB1070069 VH CDR1 | SYWMH |
| 35 | | |
| SEQ ID NO: | AB1070069 VH CDR2 | MIHPNSGSTNYNEKFNT |
| 36 | | |
| SEQ ID NO: | AB1070069 VH CDR3 | NYYAFTYEGPFDY |
| 37 | | |
| SEQ ID NO: | AB1070069 VH | QVQLQQPGAELVKPGASVKLSCKASGY |
| 38 | | TFTSYWMHWVKQRPGQGLEWIGMIHP |
| | | NSGSTNYNEKFNTKATLTVDKSSSTAY |
| | | MQLSSLTSEDSAVYYCARNYYAFTYEG |
| | | PFDYSGQGTTLTV |
| | | SS |
| SEQ ID NO: | AB1070069 VL CDR1 | KASQSVGNNVA |
| 39 | | |
| SEQ ID NO: | AB1070069 VL CDR2 | YASNRYT |
| 40 | | |
| SEQ ID NO: | AB1070069 VL CDR3 | QQHSSSPLT |
| 41 | | |
| SEQ ID NO: | AB1070069 VL | SIVMTQTPKFLPVSAGDRVTMTCKASQS |
| 42 | | VGNNVAWYQQKPGQSPKLLIYYASNRY |
| | | TGVPDRFTGSGSGTDFTFTISSVHVEDL |
| | | AVYFCQQHSSSPLTFGAGTKLELK |

1. A method for detecting one or more antigenically distinct forms of IL-33 in a biological sample, comprising:
(a) contacting the sample with one or more binding molecules each capable of binding to an antigenically distinct form of IL-33 selected from: IL-33/sST2, reduced IL-33, and/or oxidised IL-33, under conditions sufficient to form complexes; and
(b) detecting the levels of the one or more IL-33/sST2, reduced IL-33, and/or oxidised IL-33, complexes in said sample, wherein:
(i) the binding molecule which binds to IL-33/ST2 comprises a variable heavy domain having VHCDRs 1-3 of SEQ ID NOs: 17, 18 and 19, respectively, and a variable light domain having VLCDRs 1-3 of SEQ ID NOs: 21, 22 and 23, respectively wherein one or more CDRs have 3 or fewer single amino acid substitutions, deletions or insertions
(ii) the binding molecule which binds to reduced IL-33 comprises:
a. a variable heavy domain having VHCDRs 1-3 of SEQ ID NOs: 1, 2 and 3, respectively, and a variable light domain having VLCDRs 1-3 of SEQ ID NOs: 5, 6 and 7, respectively, wherein one or more CDRs have 3 or fewer single amino acid substitutions, deletions or insertions;
b. a variable heavy domain having VHCDRs 1-3 of SEQ ID NOs: 25, 26 and 27, respectively, and a variable light domain having VLCDRs 1-3 of SEQ ID NOs: 29, 30 and 31, respectively, wherein one or more CDRs have 3 or fewer single amino acid substitutions, deletions or insertions; or
c. a variable heavy domain having VHCDRs 1-3 of SEQ ID NOs: 35, 36 and 37, respectively, and a variable light domain having VLCDRs 1-3 of SEQ ID NOs: 39, 40 and 41, respectively, wherein one or more CDRs have 3 or fewer single amino acid substitutions, deletions or insertions; and
(iii) the binding molecule which binds to oxidised IL-33 comprises a variable heavy domain having VHCDRs 1-3 of SEQ ID NOs: 9, 10 and 11, respectively, and a variable light domain having VLCDRs 1-3 of SEQ ID NOs: 13, 14 and 15, respectively wherein one or more CDRs have 3 or fewer single amino acid substitutions, deletions or insertions.

2. The method according to claim 1, wherein step (a) comprises contacting the sample with the binding molecule capable of binding to IL-33/sST2, and step (b) comprises detecting the level of IL-33/sST2 complexes.

3. The method according to claim 1, wherein step (a) comprises contacting the sample with the binding molecule capable of binding to reduced IL-33, and step (b) comprises detecting the level of reduced IL-33 complexes.

4. The method according to claim 1, wherein step (a) comprises contacting the sample with the binding molecule capable of binding to oxidised IL-33, and step (b) comprises detecting the level of oxidised IL-33 complexes.

5. The method according to claim 1, wherein step (a) comprises contacting the sample with the binding molecules capable of binding to reduced IL-33 and IL-33/sST2, and step (b) comprises detecting the levels of reduced IL-33 complexes and IL-33/sST2 complexes.

6. The method according to claim 1, wherein step (a) comprises contacting the sample with the binding molecules capable of binding to oxidised IL-33 and IL-33/sST2, and step (b) comprises detecting the levels of oxidised IL-33 complexes and IL-33/sST2 complexes.

7. The method according to claim 1, wherein step (a) comprises contacting the sample with the binding molecules capable of binding to reduced IL-33 and oxidised IL-33, and step (b) comprises detecting the levels of reduced IL-33 complexes and oxidised IL-33 complexes.

8. The method according to claim 1, wherein step (a) comprises contacting the sample with the binding molecules capable of binding to reduced IL-33, and oxidised IL-33, and IL-33/sST2, and step (b) comprises detecting the levels of reduced IL-33 complexes and oxidised IL-33 complexes, and IL-33/sST2 complexes.

9. The method according to any preceding claim, wherein the one or more binding molecules are capture probes.

10. The method according to claim 9, wherein the method further comprises a step of contacting the complexes with one or more reporter molecules capable of binding to the one or more complexes.

11. The method according to any of claims 1-8, wherein, prior to step (a) the following step is carried out: immobilizing the antigenically distinct isoforms of IL-33 by contacting the sample with a solid surface under suitable conditions for the antigenically distinct isoforms of IL-33 to bind to the solid surface.

12. The method according to claim 11, wherein the detecting step (b) comprises detecting the levels of bound reporter molecules in said sample.

13. An assay kit for detecting antigenically distinct forms of IL-33 in a biological sample, comprising:
(a) A binding molecule capable of binding to an antigenically distinct form of IL-33 selected from: IL-33/sST2, reduced IL-33 and/or oxidised IL-33 to form a complex;
(b) A reporter molecule capable of binding to a binding molecule complex selected from: a IL-33/sST2, a reduced IL-33 and/or an oxidised IL-33 binding molecule complex.

14. The kit according to claim 13, wherein the binding molecule which binds to reduced IL-33 comprises:
a. a variable heavy domain having VHCDRs 1-3 of SEQ ID NOs: 1, 2 and 3, respectively, and a variable light domain having VLCDRs 1-3 of SEQ ID NOS: 5, 6 and 7, respectively, wherein one or more CDRs have 3 or fewer single amino acid substitutions, deletions or insertions;
b. a variable heavy domain having VHCDRs 1-3 of SEQ ID NOs: 25, 26 and 27, respectively, and a variable light domain having VLCDRs 1-3 of SEQ ID NOS: 29, 30 and 31, respectively, wherein one or more CDRs have 3 or fewer single amino acid substitutions, deletions or insertions; or
c. a variable heavy domain having VHCDRs 1-3 of SEQ ID NOs: 35, 36 and 37, respectively, and a variable light domain having VLCDRs 1-3 of SEQ ID NOS: 39, 40 and 41, respectively, wherein one or more CDRs have 3 or fewer single amino acid substitutions, deletions or insertions.

15. The kit according to claim 13, wherein the binding molecule which binds to oxidised IL-33 comprises a variable heavy domain having VHCDRs 1-3 of SEQ ID NOs: 9, 10 and 11, respectively, and a variable light domain having VLCDRs 1-3 of SEQ ID NOs: 13, 14 and 15, respectively, wherein one or more CDRs have 3 or fewer single amino acid substitutions, deletions or insertions.

16. The kit according to claim 13, wherein the binding molecule which binds to IL-33/ST2 comprises a variable heavy domain having VHCDRs 1-3 of: SEQ ID NOs: 17, 18 and 19, respectively, and a variable light domain having VLCDRs 1-3 of SEQ ID NOs: 21, 22 and 23, respectively, wherein one or more CDRs have 3 or fewer single amino acid substitutions, deletions or insertions.

17. The kit according to any of claims 13-16, wherein the kit comprises:
(i) An IL-33/sST2 binding molecule and a reporter molecule which binds to IL-33/sST2-binding molecule complexes; and/or
(ii) An oxidised IL-33 binding molecule and a reporter molecule which binds to oxidised-IL33-binding molecule complexes; and/or
(iii) A reduced IL-33 binding molecule and a reporter molecule which binds to reduced-IL33-binding molecule complexes.

18. An antibody or antigen binding fragment thereof which binds to reduced IL-33 comprising:
a. a variable heavy domain having VHCDRs 1-3 of SEQ ID NOs: 1, 2 and 3, respectively, and a variable light domain having VLCDRs 1-3 of SEQ ID NOS: 5, 6 and 7, respectively, wherein one or more CDRs have 3 or fewer single amino acid substitutions, deletions or insertions; or
b. a variable heavy domain having VHCDRs 1-3 of SEQ ID NOs: 25, 26 and 27, respectively, and a variable light domain having VLCDRs 1-3 of SEQ ID NOS: 29, 30 and 31, respectively, wherein one or more CDRs have 3 or fewer single amino acid substitutions, deletions or insertions; or
c. a variable heavy domain having VHCDRs 1-3 of SEQ ID NOs: 35, 36 and 37, respectively, and a variable light domain having VLCDRs 1-3 of SEQ ID NOS: 39, 40 and 41, respectively, wherein one or more CDRs have 3 or fewer single amino acid substitutions, deletions or insertions.

19. An antibody or antigen binding fragment thereof which binds to oxidised IL-33 comprising a variable heavy domain having VHCDRs 1-3 of SEQ ID NOs: 9, 10 and 11, respectively, and a variable light domain having VLCDRs 1-3 of SEQ ID NOs: 13, 14 and 15, respectively, wherein one or more CDRs have 3 or fewer single amino acid substitutions, deletions or insertions.

20. An antibody or antigen binding fragment thereof which binds to IL-33/sST2 comprising a variable heavy domain having VHCDRs 1-3 of: SEQ ID NOs: 17, 18 and 19, respectively, and a variable light domain having VLCDRs 1-3 of SEQ ID NOs: 21, 22 and 23, respectively, wherein one or more CDRs have 3 or fewer single amino acid substitutions, deletions or insertions.

21. The antibody or antigen binding fragment thereof of any of claims 18-20, conjugated to a detectable label.

22. The kit according to any of claims 13-17, the method according to any of claims 1-12 and the antibody or antigen binding fragment thereof according to claim 18, wherein the binding molecule, or antibody or antigen binding fragment thereof, which binds to reduced IL-33 comprises:
a. a variable heavy domain (VH) wherein the VH has an amino acid sequence at least 90%, for example 91, 92, 93, 94, 95, 96, 97, 98, 99 or 100% identical to a VH according to SEQ ID NO: 4, and/or a variable light domain (VL) wherein the VL has an amino acid sequence at least 90%, for example 91, 92, 93, 94, 95, 96, 97, 98, 99 or 100% identical to a VL according to SEQ ID NO: 8;
b. a variable heavy domain (VH) wherein the VH has an amino acid sequence at least 90%, for example 91, 92, 93, 94, 95, 96, 97, 98, 99 or 100% identical to a VH according to SEQ ID NO: 28, and/or a variable light domain (VL) wherein the VL has an amino acid sequence at least 90%, for example 91, 92, 93, 94, 95, 96, 97, 98, 99 or 100% identical to a VL according to SEQ ID NO: 32; or
c. a variable heavy domain (VH) wherein the VH has an amino acid sequence at least 90%, for example 91, 92, 93, 94, 95, 96, 97, 98, 99 or 100% identical to a VH according to SEQ ID NO: 38, and/or a variable light domain (VL) wherein the VL has an amino acid sequence at least 90%, for example 91, 92, 93, 94, 95, 96, 97, 98, 99 or 100% identical to a VL according to SEQ ID NO: 42.

23. The kit according to any of claims 13-17, the method according to any of claims 1-12, and the antibody or antigen binding fragment thereof according to claim 18, wherein the binding molecule, or antibody or antigen binding fragment thereof, which binds to reduced IL-33 comprises a VHCDR1 having the sequence of: SEQ ID NO:1 a VHCDR2 having the sequence of: SEQ ID NO:2 a VHCDR3 having the sequence of: SEQ ID NO: 3 a VLCDR1 having the sequence of: SEQ ID NO:5 a VLCDR2 having the sequence of: SEQ ID NO:6 and a VLCDR3 having the sequence of: SEQ ID NO:7.

24. The kit according to any of claims 13-17, the method according to any of claims 1-12, and the antibody or antigen binding fragment thereof according to claim 18, wherein the binding molecule, or antibody or antigen binding fragment thereof, which binds to reduced IL-33 comprises a VHCDR1 having the sequence of: SEQ ID NO:25 a VHCDR2 having the sequence of: SEQ ID NO:26 a VHCDR3 having the sequence of: SEQ ID NO:27 a VLCDR1 having the sequence of: SEQ ID NO:29 a VLCDR2 having the sequence of: SEQ ID NO:30 and a VLCDR3 having the sequence of: SEQ ID NO: 31.

25. The kit according to any of claims 13-17, the method according to any of claims 1-12, and the antibody or antigen binding fragment thereof according to claim 18, wherein the binding molecule, or antibody or antigen binding fragment thereof, which binds to reduced IL-33 comprises a VHCDR1 having the sequence of: SEQ ID NO: 35 a VHCDR2 having the sequence of: SEQ ID NO: 36 a VHCDR3 having the sequence of: SEQ ID NO: 37 a VLCDR1 having the sequence of: SEQ ID NO: 39 a VLCDR2 having the sequence of: SEQ ID NO: 40 and a VLCDR3 having the sequence of: SEQ ID NO: 41.

26. The kit according to any of claims 13-17, the method according to any of claims 1-12, and the antibody or antigen binding fragment thereof according to claim 19, wherein the binding molecule, or antibody or antigen binding fragment thereof, which binds to oxidised IL-33 comprises a variable heavy domain wherein the VH has an amino acid sequence at least 90%, for example 91, 92, 93, 94, 95, 96, 97, 98, 99 or 100% identical to a VH according to SEQ ID NO: 12, and/or a variable light domain wherein the VL has an amino acid sequence at least 90%, for example 91, 92, 93, 94, 95, 96, 97, 98, 99 or 100% identical to a VL according to SEQ ID NO: 16.

27. The kit according to any of claims 13-17, the method according to any of claims 1-12, and the antibody or antigen binding fragment thereof according to claim 19, wherein the binding molecule, or antibody or antigen binding fragment thereof, capable of binding to oxidised IL-33 comprises a VHCDR1 having the sequence of: SEQ ID NO:9 a VHCDR2 having the sequence of: SEQ ID NO:10 a VHCDR3 having the sequence of: SEQ ID NO:11 a VLCDR1 having the sequence of: SEQ ID NO:13 a VLCDR2 having the sequence of: SEQ ID NO: 14 and a VLCDR3 having the sequence of: SEQ ID NO: 15.

28. The kit according to any of claims 13-17, the method according to any of claims 1-12, and the antibody or antigen binding fragment thereof according to claim 20, wherein the binding molecule, or antibody or antigen binding fragment thereof, which binds to IL-33/sST2 comprises a variable heavy domain wherein the VH has an amino acid sequence at least 90%, for example 91, 92, 93, 94, 95, 96, 97, 98, 99 or 100% identical to a VH according to SEQ ID NO: 20, and/or a variable light domain wherein the VL has an amino acid sequence at least 90%, for example 91, 92, 93, 94, 95, 96, 97, 98, 99 or 100% identical to a VL according to SEQ ID NO: 24.

29. The kit according to any of claims 13-17, the method according to any of claims 1-12, and the antibody or antigen binding fragment thereof according to claim 20, wherein the binding molecule, or antibody or antigen binding fragment thereof, which binds to IL-33/sST2 comprises a VHCDR1 having the sequence of: SEQ ID NO:25 a VHCDR2 having the sequence of: SEQ ID NO:26 a VHCDR3 having the sequence of: SEQ ID NO: 27 a VLCDR1 having the sequence of: SEQ ID NO:29 a VLCDR2 having the sequence of: SEQ ID NO:30 and a VLCDR3 having the sequence of: SEQ ID NO:31.


SEQUENCE LISTING (42 sequences)

SEQ ID NO: 1 - protein, 5 aa, synthetic construct
  source 1..5: note = AB1070012 VH CDR1
TYTMH

SEQ ID NO: 2 - protein, 16 aa, synthetic construct
  source 1..16: note = AB1070012 VH CDR2
YINPYSGYTKYNQKFK

SEQ ID NO: 3 - protein, 16 aa, synthetic construct
  source 1..16: note = AB1070012 VH CDR3
DSNFSYGGIYYYSMDY

SEQ ID NO: 4 - protein, 125 aa, synthetic construct
  source 1..125: note = AB1070012 VH
PVQLQQSGAELARPGASVKMSCKASGYTFTTYTMHWVKQRPGQGLEWIGYINPYSGYTKYNQKFKDKATLTADKSSSTAYMQLSSLTSEDSAVYYCARDSNFSYGGIYYYSMDYWGQGTSVTVSS

SEQ ID NO: 5 - protein, 16 aa, synthetic construct
  source 1..16: note = AB1070012 VL CDR1
KSSQSLLDSDGKTYLN

SEQ ID NO: 6 - protein, 7 aa, synthetic construct
  source 1..7: note = AB1070012 VL CDR2
LVSKLDS

SEQ ID NO: 7 - protein, 9 aa, synthetic construct
  source 1..9: note = AB1070012 VL CDR3
WQGTHFPRT

SEQ ID NO: 8 - protein, 112 aa, synthetic construct
  source 1..112: note = AB1070012 VL
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPRTFGGGTKLEIK

SEQ ID NO: 9 - protein, 5 aa, synthetic construct
  source 1..5: note = AB1070141 VH CDR1
DYWIE

SEQ ID NO: 10 - protein, 16 aa, synthetic construct
  source 1..16: note = AB1070141 VH CDR2
EILPRSDSTNYNEKFK

SEQ ID NO: 11 - protein, 11 aa, synthetic construct
  source 1..11: note = AB1070141 VH CDR3
HYHYDGSLFVY

SEQ ID NO: 12 - protein, 120 aa, synthetic construct
  source 1..120: note = AB1070141 VH
QVQLQQSGAELMKPGASVKLSCKATGYIVTDYWIEWVKQRPGHGLEWIGEILPRSDSTNYNEKFKVRATFTADTSSNTAYMQLSSLTTEDSAIYYCASHYHYDGSLFVYWGQGTLVTVSS

SEQ ID NO: 13 - protein, 16 aa, synthetic construct
  source 1..16: note = AB1070141 VL CDR1
KSSQSLLNSRTRKNYL

SEQ ID NO: 14 - protein, 7 aa, synthetic construct
  source 1..7: note = AB1070141 VL CDR2
WASTRES

SEQ ID NO: 15 - protein, 8 aa, synthetic construct
  source 1..8: note = AB1070141 VL CDR3
KQSYSLPT

SEQ ID NO: 16 - protein, 112 aa, synthetic construct
  source 1..112: note = AB1070141 VL
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYSLPTFGGGTKLEIK

SEQ ID NO: 17 - protein, 5 aa, synthetic construct
  source 1..5: note = AB1070008 VH CDR1
SYYIH

SEQ ID NO: 18 - protein, 16 aa, synthetic construct
  source 1..16: note = AB1070008 VH CDR2
WIYPGSGNTKYNEKFK

SEQ ID NO: 19 - protein, 10 aa, synthetic construct
  source 1..10: note = AB1070008 VH CDR3
GFHYYGRMDY

SEQ ID NO: 20 - protein, 119 aa, synthetic construct
  source 1..119: note = AB1070008 VH
QVQLQQSGPELVKPGASVKTSCKASGYSFTSYYIHWVKQRPGQGLEWIGWIYPGSGNTKYNEKFKGKATLTADTSSSTAFMQLSSLTSEDSAVYYCASGFHYYGRMDYWGQGTTLTVSS

SEQ ID NO: 21 - protein, 11 aa, synthetic construct
  source 1..11: note = AB1070008 VL CDR1
RASQEISGYLS

SEQ ID NO: 22 - protein, 7 aa, synthetic construct
  source 1..7: note = AB1070008 VL CDR2
STSTLDS

SEQ ID NO: 23 - protein, 9 aa, synthetic construct
  source 1..9: note = AB1070008 VL CDR3
LQYASSPWT

SEQ ID NO: 24 - protein, 107 aa, synthetic construct
  source 1..107: note = AB1070008 VL
DIQMTQSPSSLSASLGERVSLTCRASQEISGYLSWLQQKPDGTIKRLIYSTSTLDSGVPKSFSGSRSGSDYSLTISSLESEDFADYYCLQYASSPWTFGGGTKLEIK

SEQ ID NO: 25 - protein, 5 aa, synthetic construct
  source 1..5: note = AB1070019 VH CDR1
RYWMH

SEQ ID NO: 26 - protein, 16 aa, synthetic construct
  source 1..16: note = AB1070019 VH CDR2
YINPYSGYTEYNQKFK

SEQ ID NO: 27 - protein, 15 aa, synthetic construct
  source 1..15: note = AB1070019 VH CDR3
YSYYTYDGRGYAMDY

SEQ ID NO: 28 - protein, 124 aa, synthetic construct
  source 1..124: note = AB1070019 VH
QVQLQQSGAELAKPGASVKMSCKASGYTFTRYWMHWIKQRPGQGLEWIGYINPYSGYTEYNQKFKDKATLTADKSSSTAYMQLSSLTSEDSAVYYCARYSYYTYDGRGYAMDYWGQGTSVTVSS

SEQ ID NO: 29 - protein, 11 aa, synthetic construct
  source 1..11: note = AB1070019 VL CDR1
KASQNVGTNVA

SEQ ID NO: 30 - protein, 7 aa, synthetic construct
  source 1..7: note = AB1070019 VL CDR2
SASYRYS

SEQ ID NO: 31 - protein, 9 aa, synthetic construct
  source 1..9: note = AB1070019 VL CDR3
QQYNSYPFT

SEQ ID NO: 32 - protein, 107 aa, synthetic construct
  source 1..107: note = AB1070019 VL
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPFTFGSGTKLEIK

SEQ ID NO: 33 - protein, 159 aa, Homo sapiens
  source 1..159: note = IL33 (amino acids 112-270)
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 34 - protein, 342 aa, Homo sapiens
  source 1..342: note = ST2 (HIS-tagged)
KFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPIDHHSDPAFLYKVVGAADYKDDDDKAAHHHHHHHHHH

SEQ ID NO: 35 - protein, 5 aa, synthetic construct
  source 1..5: note = AB1070069 VH CDR1
SYWMH

SEQ ID NO: 36 - protein, 17 aa, synthetic construct
  source 1..17: note = AB1070069 VH CDR2
MIHPNSGSTNYNEKFNT

SEQ ID NO: 37 - protein, 13 aa, synthetic construct
  source 1..13: note = AB1070069 VH CDR3
NYYAFTYEGPFDY

SEQ ID NO: 38 - protein, 122 aa, synthetic construct
  source 1..122: note = AB1070069 VH
QVQLQQPGAELVKPGASVKLSCKASGYTFTSYWMHWVKQRPGQGLEWIGMIHPNSGSTNYNEKFNTKATLTVDKSSSTAYMQLSSLTSEDSAVYYCARNYYAFTYEGPFDYSGQGTTLTVSS

SEQ ID NO: 39 - protein, 11 aa, synthetic construct
  source 1..11: note = AB1070069 VL CDR1
KASQSVGNNVA

SEQ ID NO: 40 - protein, 7 aa, synthetic construct
  source 1..7: note = AB1070069 VL CDR2
YASNRYT

SEQ ID NO: 41 - protein, 9 aa, synthetic construct
  source 1..9: note = AB1070069 VL CDR3
QQHSSSPLT

SEQ ID NO: 42 - protein, 107 aa, synthetic construct
  source 1..107: note = AB1070069 VL
SIVMTQTPKFLPVSAGDRVTMTCKASQSVGNNVAWYQQKPGQSPKLLIYYASNRYTGVPDRFTGSGSGTDFTFTISSVHVEDLAVYFCQQHSSSPLTFGAGTKLELK